(19) United States
(12) Patent Application Publication
(10) Pub. No.: US 2010/0249111 A1
(43) Pub. Date: Sep. 30, 2010

(54) MULTI-RING COMPOUNDS AND USES THEREOF

(75) Inventors: Norman E. Ohler, Ijamsville, MD (US); Jeffrey W. Watthey, Frederick, MD (US); Jeffrey Strovel, Laurel, MD (US); Sheela K. Chellappan, Germantown, MD (US)

Correspondence Address: Alan J. Grant;Carella Byrne Bain Gilfillan, Cecchi Stewart & Olstein, 5 Becker Farm Road, Roseland, NJ, 07068, US

(73) Assignee: AVALON PHARMACEUTICALS, Germantown, MD (US)

(21) Appl. No.: 12/451,112

(22) PCT Filed: Apr. 25, 2008
(86) PCT No.: PCT/US2008/005331

§ 371 Date: Oct. 26, 2009

Related U.S. Application Data
(60) Provisional application No. 60/926,289, filed on Apr. 26, 2007.

(51) Int. Cl. A61K 31/55; A61P 35/00; A61P 35/04; C07D 401/06; A61K 31/454; A61K 31/4545; C07D 401/14; C07D 401/12; C07D 413/10; A61K 31/5377; C07D 401/10

(52) U.S. Cl. 514/217.04; 546/200; 514/323; 514/316; 546/187; 546/194; 514/318; 544/129; 544/130; 514/235.5; 540/597

(57) ABSTRACT

Chemical agents, such as derivatives of hydroxy benzene moieties, and similar heterocyclic ring structures, including salts thereof, that act as anti-cancer and anti-tumor agents, especially where such agents modulate the activity of enzymes and structural polypeptides present in cells, such as cancer cells, or where the agents modulate levels of gene expression in cellular systems, including cancer cells, are disclosed, along with methods for preparing such agents, as well as pharmaceutical compositions containing such agents as active ingredients and methods of using these as therapeutic agents.

This application claims priority of U.S. Provisional Application No. 60/926,289, filed 26 Apr. 2007, the disclosure of which is hereby incorporated by reference in its entirety.

### FIELD OF THE INVENTION

The present invention relates to aryl and heteroaryl compounds containing multiple cyclic structural moieties and their use in modulating gene activity and in treating disease states.

### BACKGROUND OF THE INVENTION

Screening assays for novel drugs are based on the response of model cell based systems in vitro to treatment with specific compounds. Various measures of cellular response have been utilized, including the release of cytokines, alterations in cell surface markers, activation of specific enzymes, as well as alterations in ion flux and/or pH. Some such screens rely on specific genes, such as oncogenes or tumor suppressors.

The present invention utilizes screening of small molecule compounds to define groups of compounds for use as potential anticancer drugs by taking advantage of the concept that for each specific tumor type, a unique signature set of genes, that are differentially expressed in tumor cells if compared to corresponding normal: cells, can be established. Relatively small signature sets, containing 10-30 genes, allow for easy, high throughput screening for compounds that can reverse the gene expression profile from patterns typical for cancer cells to patterns seen in normal cells. The result is a number of new diverse compounds for modulating gene expression and for disease treatment, especially malignancies. A structure-activity relationship study resulted in compounds of formulas I to VI as new small molecule agents potentially having anti-neoplastic activity.

### BRIEF SUMMARY OF THE INVENTION

In one aspect, the present invention relates to small organic compounds which function as modulators, either inhibitors or agonists, of biological molecules, especially proteins and genes associated, either intimately or peripherally, with the cancerous process. The general mechanism of action of said compounds is not essential to the functioning of the present invention and such compounds are disclosed herein without limitation as to such mechanisms. In addition, the proteins and/or polypeptides that are the targets of the compounds of the invention include those that function as enzymes, such as proteases or other metabolic constituents, or that function as structural or constitutive proteins, and said target may also include oligopeptides involved in the cancerous process.

In another aspect, the present invention relates to organic compounds that function as gene expression modulators in cancer cells, especially genes involved in misregulated signal transduction pathways typical for colon cancer.

In one embodiment of the present invention, the compounds disclosed herein are able to upregulate genes found to be upregulated in normal (i.e., non-cancerous) cells versus cancer cells, especially colon cancer cells, thereby producing an expression profile for said gene(s) that resembles the expression profile found in normal cells. In another embodiment, the compounds disclosed herein are found to downregulate genes otherwise upregulated in cancer cells, especially colon cancer cells, relative to normal (i.e., non-cancerous) cells thereby producing an expression profile for said gene(s) that more resembles the expression profile found in normal cells. Thus, in addition to activity in modulating a particular gene that may or may not have a major role in inducing or sustaining a cancerous condition, the agents disclosed herein also find value in regulating a set of genes whose combined activity is related to a disease condition, such as cancer, especially colon cancer, including adenocarcinoma of the colon. Thus, while an overall set of genes is modulated, the effect of modulating any subset of these may be disproportionately large or small with respect to, the effect in ameliorating the overall disease process. Consequently, different disease conditions may rely on different subsets of genes to be active or inactive as a basis for the overall disease process. Specific gene sets are disclosed in Tables 6 and 7.

Thus, the present invention relates to novel organic compounds that have the ability to function as gene modulators for genes found in normal (i.e., non-cancer) cells and which genes are found to be upregulated or downregulated in normal cells, especially colon cells. Such an effect may prevent a disease condition, such as cancer, from arising in those otherwise more susceptible to such a condition. In one such embodiment, administration of one or more of the agents disclosed herein may succeed in preventing a cancerous condition from arising.

In other embodiments, the agents disclosed herein find use in combination with each other as well as with other agents, such as where a mixture of one or more of the agents of the present invention are given in combination or where one or more of the agents disclosed herein is given together with some other already known therapeutic agent, possibly as a means of potentiating the affects of such known therapeutic agent or vice versa.

The present invention also relates to processes of preventing or treating disease conditions, especially cancer, most especially colon cancer, by administering to a subject, such as a mammal, especially a human, a therapeutically active amount of one or more of the agents disclosed herein, including where such agents are given in combination with one or more known therapeutic agents.

### DEFINITIONS

The following terms have the indicated meaning unless expressly stated otherwise elsewhere herein.

“Acyl” is a radical formed by removal of the hydroxy from a carboxylic acid (i.e., R—C(═O)—). Preferred acyl groups include acetyl, formyl, and propionyl.

“Alkyl” is a saturated hydrocarbon chain having 1 to 15 carbon atoms, preferably 1 to 10, more preferably 1 to 5 carbon atoms (denoted herein as C₁ to C₅ alkyl or C₁-C₅ alkyl) and most preferably 1 to 4 carbon atoms. “Alkenyl” is a hydrocarbon chain having at least one (preferably only one) carbon-carbon double bond and having 2 to 15 carbon atoms, preferably 2 to 10, more preferably 2 to 5, most preferably 2 to 4 carbon atoms (denoted herein C₂ to C₄ alkenyl or C₂-C₄ alkenyl). “Alkynyl” is a hydrocarbon chain having at least one (preferably only one) carbon-carbon triple bond and having 2 to 15 carbon atoms, preferably 2 to 10, more preferably 2 to 4 carbon atoms (denoted herein C₂ to C₄ alkynyl or C₂-C₄ alkynyl). Alkyl, alkenyl and alkynyl chains (referred to collectively as “hydrocarbon chains”) may, unless expressly stated otherwise, be straight or branched and may be unsubstituted or substituted. Preferred branched alkyl, alkenyl and alkynyl chains have one or two branches, preferably one branch. Preferred chains are alkyl. Alkyl, alkenyl and alkynyl hydrocarbon chains each may be unsubstituted or substituted with from 1 to 4 substituents; when substituted, preferred chains are mono-, di-, or tri-substituted (said substituents replacing 1, 2 or 3 hydrogen atoms of the chain). Alkyl, alkenyl and alkynyl hydrocarbon chains each may be substituted with halo, hydroxy, aryloxy (e.g., phenoxy), heteroaryloxy, acyloxy (e.g., acetoxy), carboxy, aryl (e.g., phenyl), heteroaryl, cycloalkyl, heterocycloalkyl, spirocyclic substituents, amino, amido, acylamino, keto, thioketo, cyano, or any combination thereof. Preferred hydrocarbon groups include methyl, ethyl, propyl, isopropyl, butyl, tertiary-butyl (or tert-butyl) vinyl, allyl, butenyl, and exomethylenyl.

Also, as referred to herein, a “lower” alkyl, alkenyl or alkynyl moiety (e.g., “lower alkyl”) is a chain comprised of 1 to 6, preferably from 1 to 4, carbon atoms in the case of alkyl and 2 to 6, preferably 2 to 4, carbon atoms in the case of alkenyl and alkynyl.

“Alkoxy” refers to an oxygen radical having a hydrocarbon chain substituent, where the hydrocarbon chain is an alkyl, alkenyl or alkynyl (i.e., O-alkyl, —O-alkenyl or O-alkynyl). Preferred alkoxy groups include (for example) methoxy, ethoxy, propoxy and allyloxy.

“Aryl” is an aromatic hydrocarbon ring. Aryl rings are monocyclic or fused bicyclic and tricyclic ring systems. Monocyclic aryl rings contain 6 carbon atoms in the ring. Monocyclic aryl rings are also referred to as phenyl rings. Bicyclic aryl rings contain from 8 to 17 carbon atoms, preferably 9 to 12 carbon atoms, in the ring. Bicyclic aryl rings include ring systems wherein one ring is aryl and the other ring is aryl, cycloalkyl, heteroaryl, or heterocycloakyl. Preferred bicyclic aryl rings comprise 6-membered rings fused to 5-, 6-, or 7-membered rings. Aryl rings may be unsubstituted or substituted with from 1 to 4 substituents on the ring. Aryl rings may be substituted with halo, cyano, nitro, hydroxy, carboxy, amino, acylamino, alkyl, heteroalkyl, haloalkyl, phenyl, aryloxy, alkoxy, heteroalkoxy, carbamyl, haloalkyl, methylenedioxy, heteroaryloxy, or any combination thereof. Preferred aryl rings include naphthyl, tolyl, xylyl, and phenyl. The most preferred aryl ring radical is phenyl and the most preferred substitutions are halogens, alkyls and haloalkyls, most preferably —CF₃.

“Alkylaryl” or “alkaryl” is an aryl ring having an alkyl group attached thereto as a substituent, wherein the alkyl is as already defined and the aryl ring may be substituted or unsubstituted. The alkyl moiety may be single or branched chain, substituted or unsubstituted.

“Arylalkyl” or “aralkyl” is an alkyl group as defined herein with an aryl ring attached thereto as a substituent and wherein the alkyl may be straight or branched and may be substituted or unsubstituted.

“Aryloxy” is an oxygen radical having an aryl substituent (i.e., —O-aryl).

Preferred aryloxy groups include (for example) phenoxy, naphthyloxy, methoxyphenoxy, and methylenedioxyphenoxy.

“Cycloalkyl” is a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from 3 to 9 carbon atoms, preferably from 3 to 7 carbon atoms, most preferably 5 or 6 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5-, 6- or 7-membered rings fused to 5-, 6-, or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, and cyclohexyl.

“Halo” or “halogen” is fluoro, chloro, bromo or iodo. Preferred halo are fluoro, chloro and bromo; more preferred typically are chloro and fluoro, especially fluoro.

“Haloalkyl” is a straight, branched, or cyclic hydrocarbon substituted with one or more halo substituents. Preferred are C₁-C₁₂ haloalkyls; more preferred are C₁-C₆ haloalkyls; still more preferred still are C₁-C₃ haloalkyls. Preferred halo substituents are fluoro and chloro. The most preferred haloalkyl is trifluoromethyl.

“Heteroatom” is a nitrogen, sulfur, or oxygen atom, preferably nitrogen or oxygen, more preferably nitrogen. Groups containing more than one heteroatom may contain different heteroatoms.

“Heteroalkyl” is a saturated or unsaturated chain containing carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl chains have one or two branches, preferably one branch. Preferred heteroalkyl chains are saturated. Unsaturated heteroalkyl chains have one or more carbon-carbon double bonds and/or one or more carbon-carbon triple bonds. Preferred unsaturated heteroalkyl chains have one or two double bonds or one triple bond, more preferably one double bond. Heteroalkyl chains may be unsubstituted or substituted with from 1 to 4 substituents. Preferred substituted heteroalkyl chains are mono-, di-, or tri-substituted. Heteroalkyl chains may be substituted with lower alkyl, haloalkyl, halo, hydroxy, aryloxy, heteroaryloxy, acyloxy, carboxy, monocyclic aryl, heteroaryl, cycloalkyl, heterocycloalkyl, spirocyclic substituents, amino, acylamino, amido, keto, thioketo, cyano, or any combination thereof.

“Heteroaryl” is an aromatic ring containing carbon atoms and from 1 to about 6 heteroatoms in the ring. Heteroaryl rings are monocyclic or fused bicyclic ring systems. Monocyclic heteroaryl rings contain 5 or 6 member atoms, (carbon and heteroatoms) in the ring. Bicyclic heteroaryl rings contain from 8 to 17 member atoms, preferably 8 to 12 member atoms, in the ring. Bicyclic heteroaryl rings include ring systems wherein one ring is heteroaryl and the other ring is aryl, heteroaryl, cycloalkyl, or heterocycloalkyl. Preferred bicyclic heteroaryl ring systems comprise 5-, 6- or 7-membered rings fused to 5-, 6-, or 7-membered rings. Heteroaryl rings may be unsubstituted or substituted with from 1 to 4 substituents on the ring. Heteroaryl may be substituted with halo, cyano, nitro, hydroxy, carboxy, amino, acylamino, alkyl, heteroalkyl, haloalkyl, phenyl, alkoxy, aryloxy, heteroaryloxy, or any combination thereof. Preferred heteroaryl rings include, but are not limited to, the following:

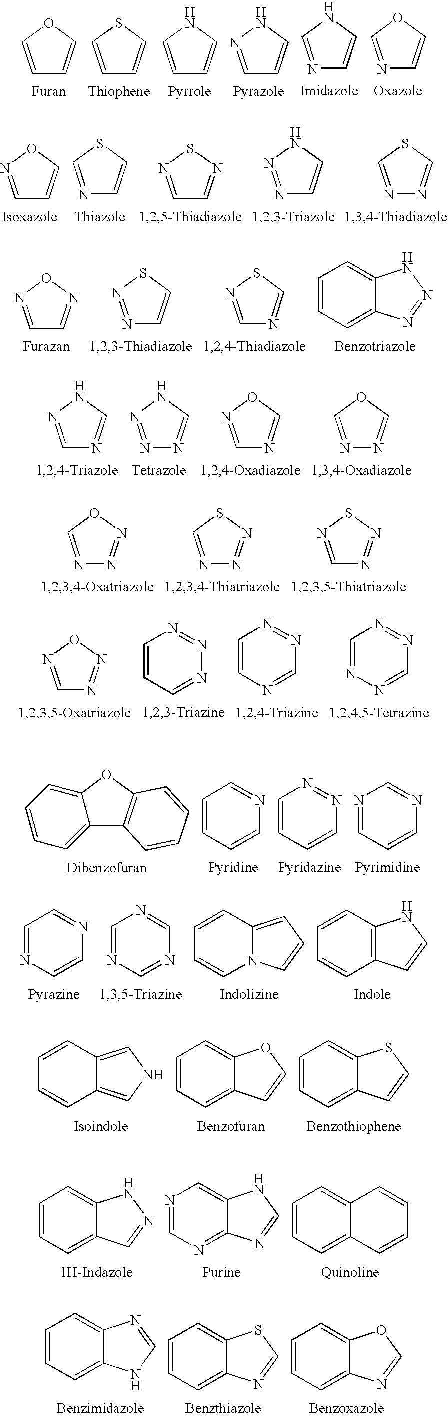

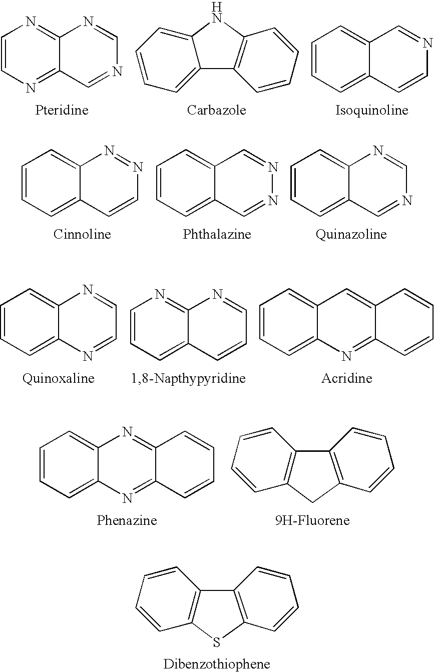

“Heteroaryloxy” is an oxygen radical having a heteroaryl substituent (i.e., —O-heteroaryl). Preferred heteroaryloxy groups include (for example) pyridyloxy, furanyloxy, (thiophene)oxy, (oxazole)oxy, (thiazole)oxy, (isoxazole)oxy, pyrmidinyloxy, pyrazinyloxy, and benzothiazolyloxy.

“Heterocycloalkyl” is a saturated or unsaturated ring containing carbon atoms and from 1 to about 4 (preferably 1 to 3) heteroatoms in the ring. Heterocycloalkyl rings are not aromatic. Heterocycloalkyl rings are monocyclic, or are fused, bridged, or spiro bicyclic ring systems. Monocyclic heterocycloalkyl rings contain from 3 to about 9 member atoms (carbon and heteroatoms), preferably from 5 to 7 member atoms, in the ring. Bicyclic heterocycloalkyl rings contain from 7 to 17 member atoms, preferably 7 to 12 member atoms, in the ring. Bicyclic heterocycloalkyl rings contain from about 7 to about 17 ring atoms, preferably from 7 to 12 ring atoms. Bicyclic heterocycloalkyl rings may be fused, spiro, or bridged ring systems. Preferred bicyclic heterocycloalkyl rings comprise 5-, 6- or 7-membered rings fused to 5-, 6-, or 7-membered rings. Heterocycloalkyl rings may be unsubstituted or substituted with from 1 to 4 substituents on the ring. Heterocycloalkyl may be substituted with halo, cyano, hydroxy, carboxy, keto, thioketo, amino, acylamino, acyl, amido, alkyl, heteroalkyl, haloalkyl, phenyl, alkoxy, aryloxy or any combination thereof. Preferred substituents on heterocycloalkyl include halo and haloalkyl. Preferred heterocycloalkyl rings include, but are not limited to, the following:

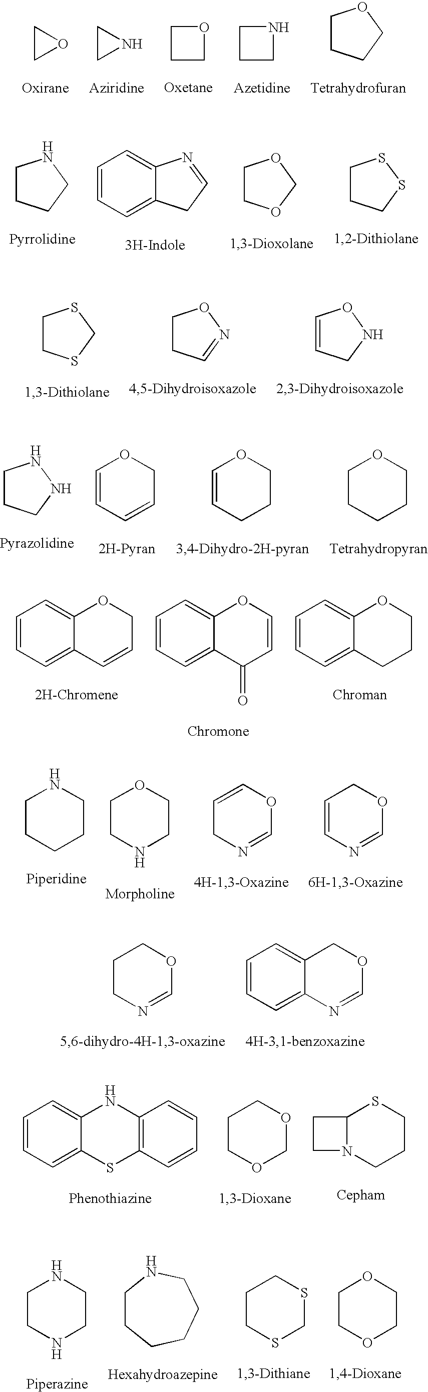

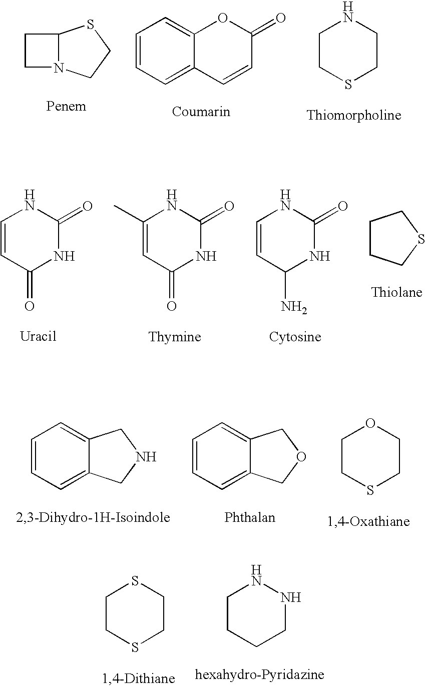

A “pharmaceutically-acceptable salt” is a cationic salt formed at any acidic (e.g., carboxylic acid) group, or an anionic salt formed at any basic (e.g., amino) group. Many such salts are known in the art, as described in World Patent Publication 87/05297, Johnston et al., published Sep. 11, 1987 incorporated by reference herein. Preferred cationic salts include the alkali metal salts (such as sodium and potassium), and alkaline earth metal salts (such as magnesium and calcium) and organic salts. Preferred anionic salts include the halides (such as chloride or hydrochloride salts), sulfonates, carboxylates, phosphates, and the like.

Such salts are well understood by the skilled artisan, and the skilled artisan is able to prepare any number of salts given the knowledge in the art. Furthermore, it is recognized that the skilled artisan may prefer one salt over another for reasons of solubility, stability, formulation ease and the like. Determination and optimization of such salts is within the purview of the skilled artisan's practice.

A “solvate” is a complex formed by the combination of a solute (e.g., a drug molecule) and a solvent (e.g., water). See J. Honig et al., The Van Nostrand Chemist's Dictionary, p. 650 (1953). Pharmaceutically acceptable solvents used according to this invention include those that do not interfere with the biological activity of the drug molecule (e.g., water, ethanol, acetic acid, N,N-dimethylformamide and others known or readily determined by the skilled artisan). When the solvent is water the complex is a hydrate.

The terms “optical isomer”, “stereoisomer”, and “diastereomer” have the accepted meanings (see, e.g., Hawley's Condensed Chemical Dictionary, 11th Ed.). The illustration of specific protected forms and other derivatives of the compounds of the instant invention is not intended to be limiting. The application of other useful protecting groups, salt forms, etc. is within the ability of the skilled artisan.

The term “metabolite” refers to a product formed from a compound of the invention by ordinary physiological processes, such as enzymatic metabolism following administration of the compound of the invention to an animal, and includes a product formed by a “prodrug” which is a chemical entity that can form a compound of the invention when administered to an animal and is then subjected to normal enzymatic and/or metabolic reactions, usually but not always catalyzed by an enzyme or by stomach acids.

Where the description of substituents (i.e., more than one R group) recites that said groups are “selected independently” or are “independently selected” this means that the two or more R groups may be either the same or different from each other.

### DETAILED SUMMARY OF THE INVENTION

In one aspect, the present invention relates to a compound having, in general, the structure of Formula I, Formula II, Formula III, Formula IV, Formula V or Formula VI:

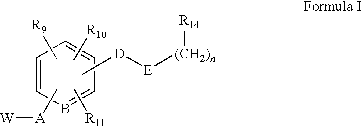

wherein W is one of

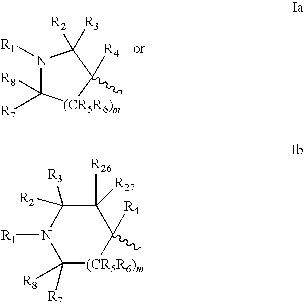

wherein when W is structure Ia, m=0, 1, 2, or 3 and n=0, 1, 2, 3, 4, or 5;
and when W is structure Ib, m=1 or 2 and n=0, 1, 2, 3, 4, or 5;
   and wherein A is selected from O and —CR₂₀R₂₁
B is selected from N and —CR₁₂;
D is selected from C═O and —CR₂₂R₂₃,
E is selected from NR₁₃ and —CR₂₄R₂₅,
such that when A is O, if D is C═O then E is —CR₂₄R₂₅ and if E is NR₁₃ then D is —CR₂₂R₂₃,

R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxyl, polyaromatic, and 5-7 ring atom heteroaryl with heteroatom N or O,
and wherein R₁ is further selected from the structure —XY,
wherein X═(CR₃₀R₃₁)ₖ (wherein k=0, 1, 2 or 3), SO₂, C═O, N R₃₀R₃₁ or —C(═O)NR₃₀, wherein R₃₀ and R₃₁ are each independently H, CH₃, or C₂ to C₅ alkyl,
and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ akenyl or C₂-C₄ alkynyl chains that may themselves be substituted or unsubstituted;
and wherein R₁₃ and R₁₄ are each further selected independently from —CHO, OR₁₅, SR₁₅, or NR₁₅R₁₆, C₁-C₄-alkyl-aryl and aryl-C₁-C₄-alkyl;

R₂, R₃, R₄, R₅, R₆, R₇, R₈, R₉, R₁₀, R₁₁, R₁₂, R₁₄, R₂₀, R₂₁ R₂₂, R₂₃, R₂₄, R₂₅, R₂₆, and R₂₇ are each independently selected from H, F, Cl, Br, I, OH, CF₃, CH₃, C₂ to C₅ alkyl, C₂ to C₅ alkenyl, C₁ to C₅ alkoxy, C₁ to C₅ hydroxyalkyl, NR₁₅R₁₆,
and wherein R₁₅ and R₁₆ are each independently selected from H, CH₃, C₂ to C₅ alkyl,

and wherein NR₁₃(CH₂)ₙR₁₄ or a portion thereof may combine to form a substituted or unsubstituted ring selected from piperidine, pyrrolidine, and tetrahydroisoquinoline,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and 0, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, SO₂NR₁₈R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In specific embodiments, n=2 and/or m=2. In other specific examples, R₉ is H, Cl or OMe. In other specific embodiments, R₁ is carbazole or diphenylethyl.

Another specific example of a compound of the invention includes a structure of Formula I wherein A is O, D is —CR₂₂R₂₃, and E is NR₁₃, preferably wherein at least one of R₂₂ and R₂₃ is H, such as wherein R₂₂ and R₂₃ are both H, or wherein one of R₂₂ and R₂₃ is OH. In another such example, A is O, D is C═O and E is CR₂₄R₂₅, such as wherein at least one of R₂₄ and R₂₅ is H, or wherein A is —CR₂₀R₂₁, D is C═O and E is NR₁₃, such as wherein R₂₀ and R₂₁ are both H or at least one of R₂₀ and R₂₁ is H. In another specific example, A is O, D is —CR₂₂R₂₃, and E is CR₂₄R₂₅, especially wherein at least one of R₂₂ and R₂₃ is hydrogen and one of R₂₄ and R₂₅ is hydrogen.

The present invention also relates to a compound having the structure of Formula II

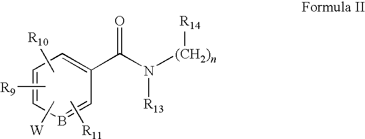

wherein W is one of

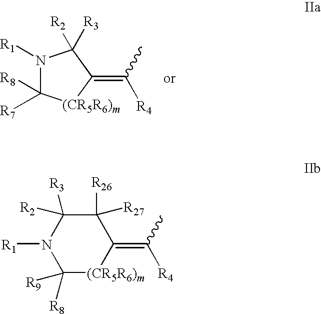

wherein when W is structure IIa, m=0, 1, 2, or 3 and n=0, 1, 2, 3, 4, or 5;

and when W is structure IIb, m=1 or 2 and n=0, 1, 2, 3, 4, or 5;

B is selected from N and —CR₁₂;

R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxyl, polyaromatic, and 5-7 ring atom heteroaryl with heteroatom N or O,
and wherein R₁ is further selected from the structure —XY,
wherein X═(CR₃₀R₃₁)ₖ (wherein k=0, 1, 2 or 3), SO₂, C═O, N R₃₀R₃₁ or —C(═O)NR₃₀, wherein R₃₀ and R₃₁ are each H, CH₃, or C₂ to C₅ alkyl,
and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ akenyl or C₂-C₄ alkynyl chains that may themselves be substituted or unsubstituted;
and wherein R₁₃ and R₁₄ are each further selected independently from —CHO, OR₁₅, SR₁₅, or NR₁₅R₁₆, C₁-C₄-alkylaryl and aryl-C₁-C₄-alkyl;
   R₂, R₃, R₄, R₅, R₆, R₇, R₈, R₉, R₁₀, R₁₁, R₁₂, R₁₄, R₂₆, and R₂₇ are each independently selected from H, F, Cl, Br, I, OH, CF₃, C₁ to C₅ alkyl, C₁ to C₅ alkenyl, C₁ to C₅ alkoxy, C₁ to C₅ hydroxyalkyl, NR₁₅R₁₆ (wherein R₁₅ and R₁₆ are each independently selected from H and C₁ to C₅ alkyl);
   and wherein R₁₅ and R₁₆ are each independently selected from H, CH₃, and C₂ to C₅ alkyl,
      and wherein NR₁₃(CH₂)ₙR₁₄ or a portion thereof may combine to form a substituted or unsubstituted ring selected from piperidine, pyrrolidine, and tetrahydroisoquinoline,
   and wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, SO₂NR₁₈R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In specific examples of such structures, R₄ is hydrogen. In other specific embodiments, R₁ is carbazole or diphenylethyl.

The present invention also relates to a compound having the structure of Formula III

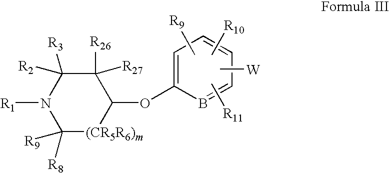

wherein W has the structure

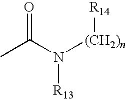

wherein m=0, 1, 2 or 3 and n=0, 1, 2, 3, 4 or 5,

and B is selected from N and —CR₁₂;
and wherein R₁ is further selected from the structure —XY,
wherein X═(CR₃₀R₃₁)ₖ (wherein k=0, 1, 2 or 3), SO₂, C═O, N R₃₀R₃₁ or —C(═O)NR₃₀, wherein R₃₀ and R₃₁ are each H, CH₃, or C₂ to C₅ alkyl,
and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ akenyl or C₂-C₄ alkynyl chains that may themselves be substituted or unsubstituted;
R₂, R₃, R₄, R₅, R₆, R₇, R₈, R₉, R₁₀, R₁₁, R₁₂, and R₁₃ are each independently selected from H, F, Cl, Br, I, OH, CF₃, CH₃, C₂ to C₅ alkyl, C₂ to C₅ alkenyl, C₂ to C₅ alkoxy, methoxyl, C₂-C₄ hydroxyalkyl, NR₁₅R₁₆,
and wherein R₁₃ is further selected from C₁ to C₅ hydroxyalkyl and —CHO,
and wherein R₁₄ is selected from H, CH₃, C₂ to C₈ alkyl, branched and unbranched C₂ to C₅ alkenyl, branched and unbranched C₂ to C₅ alkynyl, C₅ to C₇-cycloalkyl, OR₁₅, SR₁₅, —C(═O)R₁₅, —C(═O)OR₁₅, branched and unbranched (C₁ to C₅ alkyl)-NR₁₅R₁₆, NR₁₅R₁₆, branched and unbranched (C₁ to C₅ alkyl)-⁺NR₁₅R₁₆R₁₇, ⁺NR₁₅R₁₆R₁₇,
   C(═O)NR₁₅R₁₆, C(═O)ONR₁₅R₁₆, 5 to 7 membered heterocycloalkyl having up to 3 heteroatoms selected from N or O;
   aryl, heteroaryl with heteroatom N or O, aralkyl, and alkylaryl,
   and wherein each of said cycloalkyl, aryl, heteroaryl and heterocycloalkyl may be further substituted with groups each independently selected from H, F, Cl, Br, I, CF₃, branched and unbranched C₁ to C₅ alkyl, branched and unbranched C₁ to C₅ alkenyl, branched and unbranched C₁ to C₅ alkynyl, branched and unbranched C₁ to C₅ alkoxy, branched and unbranched —C₁ to C₅ alkylamino, branched and unbranched —C₁ to C₅ aminoalkyl, —C(═O)R₁₅, —C(═O)R₂₁, C(═O)OR₁₅, C(═O)OR₂₁, C₅ to C₇-cycloalkyl, —OR₁₅, —SR₁₅, wherein each of said alkyl, alkenyl, alkynyl, alkoxyl, alkylamino and amino alkyl groups may be further substituted with one or more of methyl, ethyl, n-propyl, isopropyl, n-butyl, sec-butyl, isobutyl or tert-butyl;
   and wherein R₁₅ and R₁₆ are each independently selected from H, C₁ to C₅ alkyl, C₁ to C₅ alkyl-R₂₁, C₂ to C₅ alkenyl, substituted or unsubstituted phenyl, —C(═O)R₁₉, —C(═O)OR₁₉, (C₁ to C₅ alkyl)-OH, (C₁ to C₅ alkyl)-NR₁₉R₂₀, —NR₁₉R₂₀,
   C(═O)—NR₁₉R₂₀ (wherein each of said R₁₉ and R₂₀ is independently H, methyl, ethyl, n-propyl, isopropyl, n-butyl, sec-butyl, isobutyl or tert-butyl;),
   and wherein R₂₁ is selected from 5 to 7 membered cycloalkyl, 5 to 7 membered aryl, 5 to 7 membered heteroaryl, and 5 to 7 membered heterocycloalkyl, wherein said heteroatom is N or O, each of which may be substituted with groups selected from R₁₅,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In specific examples of such compounds, R₂₀ and R₁ are each hydrogen or at least one of R₂₀ and R₁ is hydrogen.

The present invention further relates to a compound having the structure of Formula IV

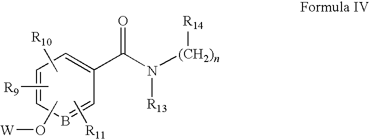

wherein W is one of

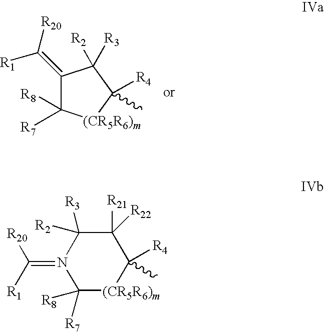

wherein when W is structure IVa, m=0, 1, 2, or 3 and n=0, 1, 2, 3, 4, or 5;

and when W is structure IVb, m=1 or 2 and n=0, 1, 2, 3, 4, or 5;

B is selected from N and —CR₁₂;

R₁₃ and R₁₄ are each independently selected from
H, CH₃, C₂ to C₅ alkyl, C₂ to C₅ alkenyl, C₁ to C₅ alkoxy, 5-9 ring atom cycloalkyl,
OR₁₅, SR₁₅, or NR₁₅R₁₆;
5-9 ring atom heterocycloalkyl having up to 3 heteroatoms selected from N or O and wherein when said heteroatom is N, it may be further substituted as may any carbon in said ring;
5-7 ring atom aryl, aryloxyl, polyaromatic, 5-7 ring atom heteroaryl with heteroatom selected from N and O, aralkyl and alkylaryl;
R₁, R₂, R₃, R₄, R₅, R₆, R₇, R₈, R₉, R₁₀, R₁₁, R₁₂, R₁₄, R₂₀, R₂₁ and R₂₂ are each independently selected from H, F, Cl, Br, I, OH, CF₃, C₁ to C₅ alkyl, C₁ to C₅ alkenyl, C₁ to C₅ alkoxy, NR₁₅R₁₆;
and wherein R₁₅ and R₁₆ are each independently selected from H, CH₃ and C₂ to C₅ alkyl
   and wherein NR₁₃(CH₂)ₙR₁₄ or a portion thereof may combine to form a substituted or unsubstituted ring selected from piperidine, pyrrolidine, and tetrahydroisoquinoline,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In preferred embodiments of any of the formulas of the invention, where R₁ is 4-phenylphenyl, carbazole, dibenzothiophene, dibenzofuran, or fluorene, each is substituted at the 2 or 3 position and where R₁ is carbazole, the latter is preferably substituted at the nitrogen thereof.

In specific examples of these compounds, R₂₀ and R₂₁ are each hydrogen or at least one of R₂₀ and R₂₁ is hydrogen.

The present invention further relates to a compound having the structure of Formula V

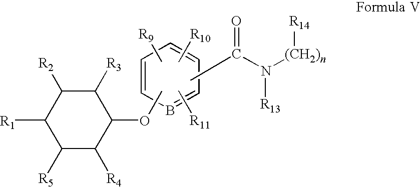

wherein n=0, 1, 2, 3, 4 or 5,

wherein B is selected from N and —CR₁₂;
R₁₃ and R₁₄ are each independently selected from H, C₁ to C₅ alkyl, C₁ to C₅ alkenyl, C₁ to C₅ alkoxy, 5-9 ring atom cycloalkyl,
OR₁₅, SR₁₅, or NR₁₅R₁₆;
5-9 ring atom heterocycloalkyl having up to 3 heteroatoms selected from N or O and wherein when said heteroatom is N, it may be further substituted as may any carbon in said ring;
5-7 ring atom aryl, aryloxy, polyaromatic, 5-7 ring atom heteroaryl with heteroatom N or O,
aralkyl and alkylaryl;
R₁, R₂, R₃, R₄, R₅, R₉, R₁₀, R₁₁, R₁₂, R₂₀, R₂₁ R₂₂, R₂₃, R₂₄, R₂₅, R₂₆, and R₂₇ are each independently selected from H, F, Cl, Br, I, OH, CF₃, CH₃, C₂ to C₅ alkyl, ═CH—, C₂ to C₅ alkenyl, C₁ to C₅ alkoxy, NR₁₅R₁₆, and wherein R₁₅ and R₁₆ are each independently selected from H, CH₃ and C2 to C₅ alkyl

nd wherein NR₁₃(CH₂)ₙR₁₄ or a portion thereof may combine to form a substituted or unsubstituted ring selected from piperidine, pyrrolidine, tetrahydroisoquinoline, and piperazine,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-7 ring atom cycloalkyl, 5-7 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In specific examples of such compounds, R₂₀ and R₂₁ are each hydrogen or at least one of R₂₀ and R₂₁ is hydrogen. In other specific embodiments, R₁ is carbazole or diphenylethyl.

The present invention also relates to a compound having the structure of Formula VI:

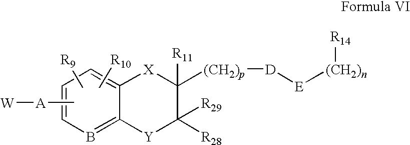

wherein W is one of

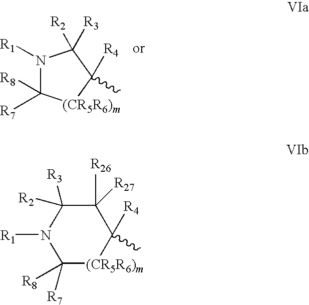

wherein when W is structure VIa, m=0, 1, 2, or 3 and n=0, 1, 2, 3, 4, or 5;

and when W is structure VIb, m=1 or 2 and n=0, 1, 2, 3, 4, or 5;

wherein p=0, 1, 2, or 3;

and wherein A is selected from O and —CR₂₀R₂₁,
B is selected from N and —CR₁₂;
D is selected from C═O and —CR₂₂R₂₃,
E is selected from NR₁₃ and —CR₂₄R₂₅,
X═(CR₃₀R₃₁)_{q}, wherein q=0, 1 or 2,
Y═(CR₃₂R₃₃)ᵣ, wherein r=0, 1 or 2

R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxy, polyaromatic, and 5-7 ring atom heteroaryl with heteroatom N or O, aralkyl and alkylaryl,
and wherein R₁ is further selected from the structure —XY,
wherein X═(CR₃₀R₃₁)ₖ (wherein k=0, 1, 2 or 3), SO₂, C═O, N R₃₀R₃₁ or —C(═O)NR₃₀, wherein R₃₀ and R₃₁ are each H, CH₃, or C₂ to C₅ alkyl,
and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ akenyl or C₂-C₄ alkynyl chains that may themselves be substituted or unsubstituted;
and wherein R₁₃ and R₁₄ are each further selected independently from —CHO, OR₁₅, SR₁₅, or NR₁₅R₁₆

R₂, R₃, R₄, R₅, R₆, R₇, R₈, R₅, R₁₀, R₁₁, R₁₂, R₂₀, R₂₁ R₂₂, R₂₃, R₂₄, R₂₅, R₂₆, R₂₇, R₂₈, R₂₉, R₃₀, R₃₁, R₃₂ and R₃₃ are each independently selected from H, F, Cl, Br, I, OH, CF₃, C₁ to C₅ alkyl, C₁ to C₅ alkenyl, C₁ to C₅ alkoxy, NR₁₅R₁₆

wherein R₁₅ and R₁₆ are each independently selected from H, CH₃ and C₂ to C₅ alkyl;
and wherein NR₁₃(CH₂)ₙR₁₄ or a portion thereof may combine to form a substituted or unsubstituted ring selected from piperidine, pyrrolidine, tetrahydroisoquinoline, and piperazine,
wherein any of said R groups (of any of the formulas) may be substituted or unsubstituted, wherein said substitutions are each independently selected from hydrogen, CH₃, hydroxyl, sulfhydryl, alkoxy; thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;

including all pharmaceutically acceptable salts, derivatives, prodrugs, metabolites, solvates, hydrates, and isomers thereof.

In a preferred embodiment of Formula VI, each of R₂, R₃, R₄ and R₅ is hydrogen. In other specific examples n=2, and/or R₉ is H, Cl or OMe and/or R₁₃ is H.

In preferred embodiments of Formulas I to VI, where R₁ is a structure comprising up to 3 fused or unfused rings, said rings are chosen from 4-phenylphenyl, carbazole, dibenzothiophene, dibenzofuran, fluorene, phenyl or naphthyl. Each of the latter may be substituted, especially with lower alkyl groups, preferably methyl or ethyl, and each ring may be separated from the remainder of the molecule by an alkyl chain of one or two carbons. Very preferred embodiments of these are N-methylcarbazole and N-ethylcarbazole, especially wherein these are separated from the remainder of the molecule by at least a methylene group, such as where said methylene is attached to the nitrogen of a piperidine ring.

The invention also contemplates pharmaceutical compositions of any of these, said compositions comprising a therapeutically effective amount of such compound in a pharmaceutically acceptable carrier. The invention further relates to a method of preventing, treating or ameliorating cancer or tumor metastasis in a mammal comprising administering to said mammal an effective amount of such compound or composition.

These embodiments thus contemplate structures wherein the central benzene or pyridine ring is fused to a cycloalkyl ring, with the benzene ring the preferred embodiment. Specific but non-limiting examples of such embodiments have the formula of one of the following:

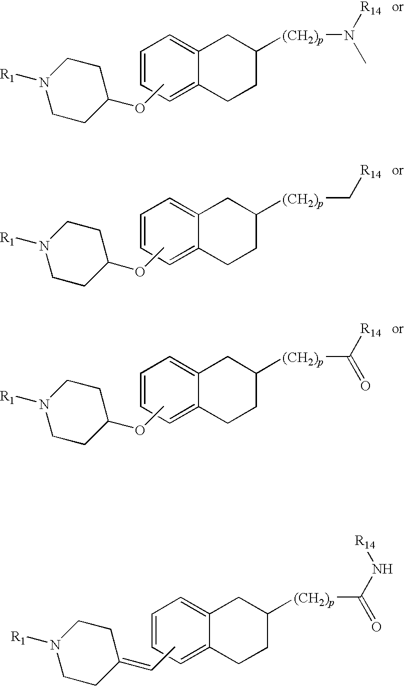

wherein p=0, 1, 2 or 3, R₁ has the range of structures recited in Tables 1 to 3, and wherein some but not all preferred embodiments of these structures are found in Table 4.

In other preferred embodiments of the structures of the invention, the substituent attached to the ring nitrogen, for example, the ring nitrogen of piperidine or pyrrolidine (e.g., R₁ in the structures of Tables 1, 2 and 3), is a diphenylethyl or carbazole group, the latter of which is preferably separated from said ring nitrogen by at least one or more methylene groups, preferably 1 or 2 methylene groups and wherein when said carbazole is substituted, it is substituted at the ring nitrogen thereof, preferably with one of H, lower alkyl or benzyl. In all of the embodiments of the invention, where an aryl, alkaryl or aralkyl group is recited, a substituted or unsubstituted phenyl group is the preferred aryl moiety of said substituent.

The present invention does not encompass embodiments of the formulas wherein the atoms of NR₁₃(CH₂)ₙR₁₄ (such as where E is NR₁₃ in structures of Formula I) combine to form a piperazine ring.

In additional preferred embodiments, NR₁₃(CH₂)ₙR₁₄ is selected from N,N-dialkyl, N-alkyl-N-alkenyl, N-alkyl-N-alkylaminoalkyl and N-alkyl-N-alkoxyalkyl. Further preferred embodiments include compounds combining any or all of these preferred embodiments as structural limitations.

In any of the structures of the invention, R₁₄ may be selected from any of H, C₁ to C₅ alkyl, C₁ to C₅ alkenyl, C₁ to C₅ alkoxy, cycloalkyl, OR₁₅, SR₁₅, or NR₁₅R₁₆ (wherein R₁₅ and R₁₆ are each independently selected from H and C₁ to C₅ alkyl); heterocycloalkyl having up to 3 heteroatoms selected from N or O and wherein when said heteroatom is N, it may be further substituted as may any carbon in said ring; aryl, aryloxy, polyaromatic, heteroaryl with heteroatom N or O, aralkyl and alkylaryl; as well as F, Cl, Br, I, OH, CF₃, NR₁₅R₁₆ (wherein R₁₅ and R₁₆ are each independently selected from H and C₁ to C₅ alkyl); wherein it may be substituted or unsubstituted, with substitutions selected from hydrogen, methyl, hydroxyl, sulfhydryl, alkoxy, thioalkoxy, alkyl, halogen, CN, CF₃, NO₂, cycloalkyl, heterocycloalkyl, aryl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, and aryl may be further substituted with a group selected from R₂ as described elsewhere herein.

In one embodiment, the present invention relates to compounds having the general structure of one of the following (wherein each R group has the meaning recited elsewhere herein for the structures of the invention):

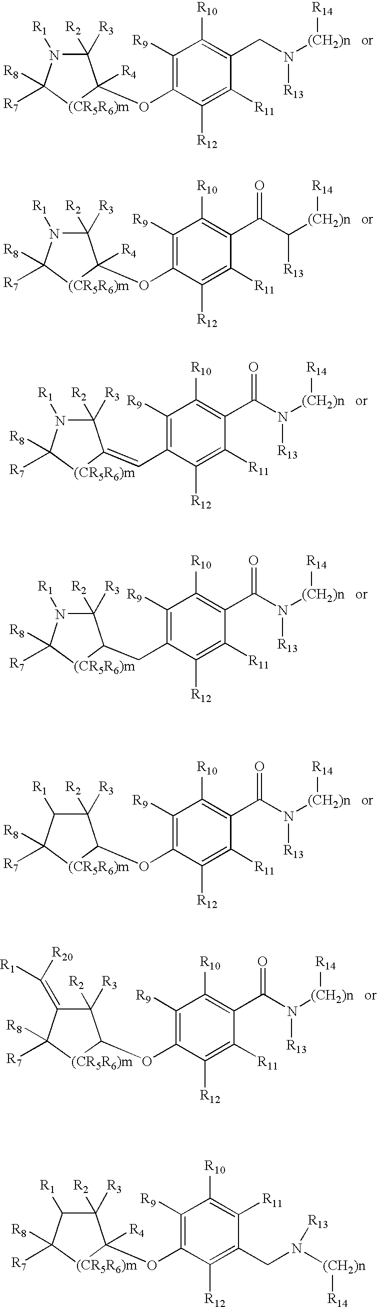

In a highly preferred embodiment, the compounds of the invention are those with structures found in Table 1.

In a highly preferred embodiment, the compounds of the invention are those with structures found in Table 2.

In a highly preferred embodiment, the compounds of the invention are those with structures found in Table 3.

In a highly preferred embodiment, the compounds of the invention are those with structures found in Table 4.

In a highly preferred embodiment, the compounds of the invention are those with structures found in Table 5.

In a highly preferred embodiment, the compounds of the invention are those with structures identified as S1-S11.

In another aspect, the present invention relates to compositions of any of the compounds of the invention, preferably wherein such compound is present in a pharmaceutically acceptable carrier and in a therapeutically effective amount. Such compositions will generally comprise an amount of such compound that is not toxic (i.e., an amount that is safe for therapeutic uses).

In accordance with the foregoing, the present invention is directed to use of the compounds of the invention as active ingredients for medicaments, in particular for medicaments useful for the treatment of tumors. The compounds of the invention will thus be present in pharmaceutical compositions containing compounds of formulas I to V as active ingredients, in admixture with pharmaceutically acceptable vehicles and excipients, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition). Use of such carriers is well known to those skilled in the art and will not be discussed further herein.

Also in accordance with the foregoing, the present invention relates to a method for preventing or treating a disease associated with a change in levels of expression of particular sets of genes in a mammal comprising administering to said mammal an effective amount of a compound of the invention.

Compounds according to the present invention will have the effect of reducing size and number of tumors, especially primary tumors, in a mammal, especially a human, in need of such treatment. A statistically significant change in the numbers of primary tumor or metastasizing cells will typically be at least about 10%, preferably 20%, 30%, 50%, 70%, 90%, or more.

In accordance with the present invention, the agents described herein may be combined with other treatments of the medical conditions described herein, such as other chemotherapies, radiation treatments, immunotherapy, surgical treatments, and the like. The compounds of the invention may also be administered in combination with such other agents as painkillers, diuretics, antidiuretics, antivirals, antibiotics, nutritional supplements, anemia therapeutics, blood clotting therapeutics, bone therapeutics, and psychiatric and psychological therapeutics.

Determination of the appropriate treatment dose is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects.

The phrase “effective amount” means an amount sufficient to effect a desired response, or to ameliorate a symptom or sign, e.g., of metastasis or primary tumor progression, size, or growth. Typical mammalian hosts will include mice, rats, cats, dogs, and primates, including humans. An effective amount for a particular patient may vary depending on factors such as the condition being treated, the overall health of the patient, the method, route, and dose of administration and the severity of side affects. Preferably, the effect will result in a change in quantitation of at least about 10%, preferably at least 20%, 30%, 50%, 70%, or even 90% or more. When in combination, an effective amount is in ratio to a combination of components and the effect is not limited to individual components alone.

An effective amount of a therapeutic will modulate the symptoms typically by at least about 10%; usually by at least about 20%; preferably at least about 30%; or more preferably at least about 50%. Alternatively, modulation of migration will mean that the migration or trafficking of various cell types is affected. Such will result in, e.g., statistically significant and quantifiable changes in the numbers of cells being affected. This may be a decrease in the numbers of target cells being attracted within a time period or target area. Rate of primary tumor progression, size, or growth may also be monitored.

In another aspect, the present invention relates to a method for preventing or treating a disorder modulated by altered gene expression, wherein the disorder is selected from the group consisting of cancer, cardiovascular disorders, arthritis, osteoporosis, inflammation, periodontal disease and skin disorders, comprising administering to a mammal in need of such treatment or prevention a therapeutically effective amount of a compound of the invention.

In a preferred embodiment thereof, the disorder is cancer, more preferably colon cancer, most preferably adenocarcinoma, and the treatment prevents, arrests or reverts tumor growth, metastasis or both.

In a preferred embodiment, the present invention relates to a method of preventing, treating or ameliorating cancer or tumor metastasis in a mammal comprising administering to said mammal an effective a compound of the invention, preferably where said mammal is a human.

The compounds of the invention will commonly exert a therapeutic effect by modulation of one or more genes found in a cell, especially a mammalian cell, such as a cancer cell, preferably colon cancer and most preferably adenocarcinoma. Thus, a compound, or compounds, of the invention can be used to determine or demarcate a set of genes by determining modulation of such set of genes by one or more compounds of the invention. For example, where a set of genes is found to be up regulated in cancer cells versus otherwise normal cells, especially normal cells of the same tissue or organ as the cancer cells, a set of genes can be determined by their common property of being modulated (based on a change in expression of the genes, such as a change in rate or amount of RNA transcribed or the amount of polypeptide produced by said expression) by contacting such genes, or a cell containing such genes, with one or more of the compounds of the invention. The extent of such modulation may, of course, be related to the amount of said compound, or compounds, used in the contacting. Such modulation may include the increased expression of all the determined genes (i.e., the genes of the set), the decreased expression of all genes of the set, or the increase in expression of some of the genes of the set and decreased expression of others. Thus, a gene not modulated by the test compound (the compound used in contacting the genes or cell containing them) is not considered a member of the set.

Thus, the present invention relates to a gene set wherein expression of each member of said gene set is modulated as a result of contacting said gene set with a compound of the invention. In specific embodiments, expression of each member of said gene set is increased as a result of said contacting or is decreased as a result of said contacting. In another preferred embodiment, the gene set is present in a cell. Such a gene set will commonly be related to a specific disease process, such as a set of genes all of which are modulated by a compound of the invention wherein such compound has a specific therapeutic effect, such as being an anti-neoplastic agent.

In another aspect, the present invention relates to a method for identifying an agent that modulates the expression of a gene set of the invention, comprising:

(a) contacting, or otherwise using, a compound, such as a test compound, a test system, such as a source of genes or polynucleotides, for example, those found to be related to a given disease or disorder, or a set that is modulated by a given compound, or group of compounds, especially where these are found in a cell, so that the cell represents the test system, containing one or more polynucleotides corresponding to each of the members of the gene set of the invention under conditions wherein the members of said gene set are being expressed;

(b) determining a change in expression of each of said one or more polynucleotides of step (a) as a result of said treatment;

wherein said change in expression of step (b) indicates modulation of the members of said gene set by the test compound thereby identifying a test compound that modulates the expression of said gene set.

In one embodiment, the cell is a naturally derived cell that contains genes of a gene set or may be a recombinant cell engineered to comprise the genes or polynucleotides of the gene set. In an alternative embodiment, the test system may comprise the genes or polynucleotides in a cell-free system.

In a related aspect, the present invention provides a method for identifying a test compound that modulates the expression of a gene set, such as a gene set of the invention, comprising:

(a) contacting a test compound with one or more polynucleotides corresponding to each of the members of the gene set of the invention under conditions wherein the members of said gene set are being expressed;

(b) determining a change in expression of each of said one or more polynucleotides of step (a) as a result of said contacting;

wherein said change in expression of step (b) indicates modulation of the members of said gene set thereby identifying a test compound that modulates the expression of said gene set.

As used herein, “corresponding genes” or “corresponding polynucleotides” or “polynucleotides corresponding to genes” refers to polynucleotides and/or genes that encode an RNA that is at least 90% identical, preferably at least 95% identical, most preferably at least 98% identical, and especially identical, to an RNA encoded by one of the genes disclosed herein in Tables 4 and 5. Such genes will also encode the same polypeptide sequence, but may include differences in such amino acid sequences where such differences are limited to conservative amino acid substitutions, such as where the same overall three-dimensional structure, is maintained. A “corresponding gene” includes splice variants thereof.

The polynucleotides useful in the methods of the invention may be genomic in nature and thus represent the sequence of an actual gene, such as a human gene, or may be a cDNA sequence derived from a messenger RNA (mRNA) and thus represent contiguous exonic sequences derived from a corresponding genomic sequence, or they may be wholly synthetic in origin for purposes of practicing the processes of the invention. Because of the processing that may take place in transforming the initial RNA transcript into the final mRNA, the sequences disclosed herein may represent less than the full genomic sequence. They may also represent sequences derived from ribosomal and transfer RNAs. Consequently, the gene as present in the cell (and representing the genomic sequence) and the polynucleotide transcripts disclosed herein, including cDNA sequences, may be identical or may be such that the cDNAs contain less than the full genomic sequence. Such genes and cDNA sequences are still considered “corresponding sequences” (as defined elsewhere herein) because they both encode the same or related RNA sequences (i.e., related in the sense of being splice variants or RNAs at different stages of processing). Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous cells (here, breast cancer) and are used to determine gene activity or expression because they represent the same sequence or are complementary to RNAs encoded by the gene. Such a gene also includes different alleles and splice variants that may occur in the cells used in the methods of the invention, such as where recombinant cells are used to assay for anti-neoplastic agents and such cells have been engineered to express a polynucleotide as disclosed herein, including cells that have been engineered to express such polynucleotides at a higher level than is found in non-engineered cancerous cells or where such recombinant cells express such polynucleotides only after having been engineered to do so. Such engineering includes genetic engineering, such as where one or more of the polynucleotides disclosed herein has been inserted into the genome of such cell or is present in a vector.

Such cells, especially mammalian cells, may also be engineered to express on their surfaces one or more of the polypeptides of the invention for testing with antibodies or other agents capable of masking such polypeptides and thereby removing the cancerous nature of the cell. Such engineering includes both genetic engineering, where the genetic complement of the cells is engineered to express the polypeptide, as well as non-genetic engineering, whereby the cell has been physically manipulated to incorporate a polypeptide of the invention in its plasma membrane, such as by direct insertion using chemical and/or other agents to achieve this result.

In a preferred embodiment of such method, the determined change in expression is a decrease in expression of said one or more polynucleotides or a decrease in said expression. In other preferred embodiments, the determined change in expression is a change in transcription of said one or more polynucleotides or a change in activity of a polypeptide, or expression product, encoded by said polynucleotide, including a change in the amount of said polypeptide synthesized, such as by a cell. The term “expression product” means that polypeptide or protein that is the natural translation product of the gene and any nucleic acid sequence coding equivalents resulting from genetic code degeneracy and thus coding for the same amino acid(s).

In additional preferred embodiments, said one or more polynucleotides are present in a cell, preferably a cancer cell, more preferably a colon cancer cell, and most preferably where the colon cancer cell is an adenocarcinoma cancer cell. In another preferred embodiment of the invention, the cell is a recombinant cell engineered to contain said set of genes.

Such methods serve to identify other compounds that have like activity, including expected therapeutic activity, as the compounds of the invention and thus serve as the basis for large scale screening assays for therapeutic compounds. As a result, one or more compounds of the invention can be utilized to determine the presents of gene sets and subsets within the genome of a cell. Thus, the set of all genes modulated by a group of structurally related compounds of the invention can form a gene set while the different sets of genes regulated by each compound of a group will form a subset. By way of non-limiting example, where a structurally related group of 5 of the compounds of the invention (all having generally the structure of Formula I) modulate (by increasing or decreasing) expression of determined genes 1-20, this latter group of genes forms a gene set. Further examination then determines that genes 1-6 are modulated by compound A, genes 7-10 are modulated by compound B, genes 2-4 and 9-12 are modulated by compound C, genes 10-20 are modulated by compound D and the even numbered genes are modulated by compound E. Each of these groups of genes, such as the genes modulated by compound C, is considered a subset of the gene set of genes 1-20. In an analogous manner, the genes modulated by compound E can be themselves further subdivided into at least 2 subsets wherein one subset is made up of the genes whose expression is increased by compound E while the other subset is made up of genes whose expression is decreased by compound E, thus yielding subsets of subsets. It should be noted that within the context of the present invention, it is not necessary to identify subsets and that each so-called subset is, in its own right, a gene set as used in the invention. The identification of sets and subsets is thus a function of the extent that a user of the methods of the invention wishes to determine modulation of genes resulting from contacting of one or more compounds of the invention. Thus, the genes modulated by a single compound form a gene set and it is not necessary, in carrying out the methods of the invention, to compare different groups of genes for modulation by more than one compound but this may, of course, be done.

In accordance with the foregoing, the present invention relates to a set of genes comprising a plurality of subsets of genes wherein each subset of said plurality is a gene set identified by the methods of the invention. The present invention also relates to compounds identified as having activity using the methods of the invention, such as novel compounds not specifically described herein by structure but which have been identified by their ability to modulates one or more gene sets modulated by compounds of the invention.

In a preferred embodiment, the present invention encompasses the gene sets and subsets of the genes identified in Table 6 and/or in Table 7. In using the compounds of the invention for treatment of disease, especially cancer, the present invention specifically contemplates use of a compound that modulates the expression of a set of, or subset of, genes of Table 7.

The present invention also comprises methods for the preparation of compounds of the invention.

### Compound Preparation:

The compounds of the invention can be prepared using a variety of procedures known in the art. The starting materials used in preparing the compounds of the invention are known, made by known methods, or are commercially available. Particularly preferred syntheses are described in the following general reaction schemes.

Examples of compounds made for these inventions are mentioned below and compounds for which no preparation is given can be made by methods known in the literature or are of common knowledge by skilled artisan.

The skilled artisan will recognize that some reactions are best carried out when another potentially reactive functionality on the molecule is masked or protected, thus avoiding any undesirable side reactions and/or increasing the yield of the reaction. Often protecting groups are used to accomplish such increased yields or to avoid the undesired reactions. Such reactions are well within the ability of the skilled artisan. Some examples are found in T. Greene, Protecting Groups in Organic Synthesis.

### EXAMPLES

### Example 1

### a) Diethyl biphenyl-4-ylmethylphosphonate

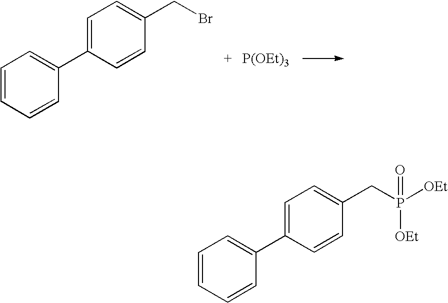

A mixture of 4-(bromomethyl) biphenyl (4.0 g, 16.2 mmol) and triethyl phosphite (3.5 g, 21 mmol) was stirred at 100° C. for 2 h and at 150° C. for 24 h under argon. The clear solution became colorless solid after cooling at room temperature. The product was obtained in quantitative yield and used as such for the next step.

### b) Methyl-3-chloro-4-(4-oxocyclohexyloxy)benzoate

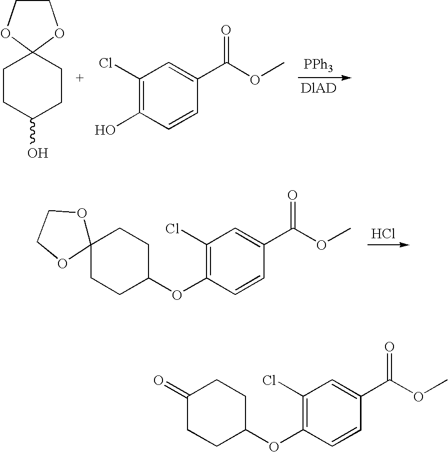

To a mixture of 1,4-dioxaspiro[4.5]decan-8-ol (2.54 g, 16 mmol) and methyl 3-chloro-4-hydroxybenzoate (2.5 g, 13.4 mmol) in anhydrous THF (40 ml) at room temperature was added triphenylphosphine (4.2 g, 16 mmol). DIAD (3.1 ml, 16 mmol) in THF (10 ml) was added dropwise over a 20 min period and the reaction mixture was stirred overnight at room temperature. The reaction was quenched by addition of water and the mixture was extracted with ethyl acetate. The combined organic extracts were washed with 1N HCl, followed by water and brine, dried (MgSO₄), filtered and concentrated under vacuum. The crude product was purified by flash column chromatography (20% EtOAc in hexane) to give the product as a colorless thick oil (4.4 g). The ketal was dissolved in THF (10 ml) and hydrolyzed using 5% HCl (15 ml), stirred overnight. Normal aqueous work up followed by extraction with EtOAc gave the crude cyclohexanone derivative which was purified further by flash column chromatography (40% EtOAc in Hexane) to give a colorless viscous product (2.75 g, 73%).

### c) Methyl 4-(4-(biphenyl-4-ylmethylene)cyclohexyloxy)-3-chlorobenzoate

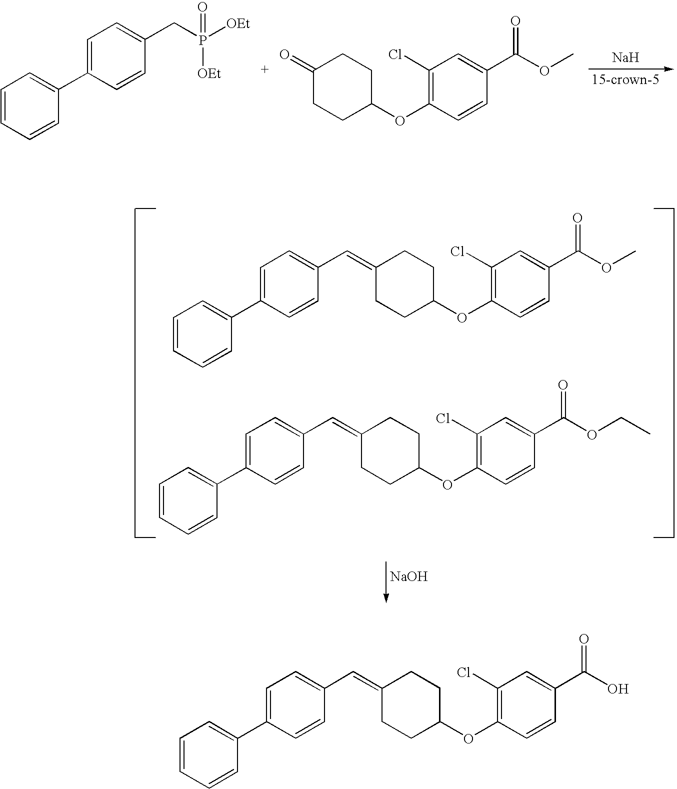

To an ice-cooled stirred solution of diethyl biphenyl-4-ylmethylphosphonate (1.5 g, 4.9 mmol) and 15-crown-5 (0.070 ml, 0.35 mmol) in THF (10 ml) was added NaOH (95% in oil, 125 mg, 4.9 mmol) and the mixture was stirred at 0° C. for 30 min. To the mixture was added a solution of methyl 3-chloro-4-(4-oxocyclohexyloxy)benzoate (1 g, 3.5 mmol) in THF (3 ml) dropwise over 10 min. at 0° C., and the mixture was stirred at room temperature for 4 h. The mixture was poured into ice water and extracted with EtOAc. The organic layer was washed with saturated aqueous NaHCO₃ and brine, dried, filtered and concentrated in vacuo. The residue was purified by flash column chromatography to give a mixture of methyl and ethyl esters. (625 mg); this was used as such for the hydrolysis step.

### d) 4-(4-(biphenyl-4-ylmethylene)cyclohexyloxy)-3-chlorobenzoic acid

To a solution of the above ester (625 mg) in a mixture of THF: MeOH(20:5) was added 50% aqueous NaOH (2.82 g, 0.35 mmol) and the mixture was stirred at room temperature overnight. The mixture was diluted with EtOAc and acidified with 1N HCl (50 ml) at room temperature. The organic layer was separated, and the aqueous layer was extracted with EtOAc. The combined organic layer was dried (MgSO₄), filtered and concentrated in vacuo. The colorless product got precipitated and filtered and dried to give the corresponding acid (580 mg).

### e) 4-(4-(biphenyl-4-ylmethylene)cyclohexyloxy)-3-chloro-N-(2-diethylamino)ethylbenzamide

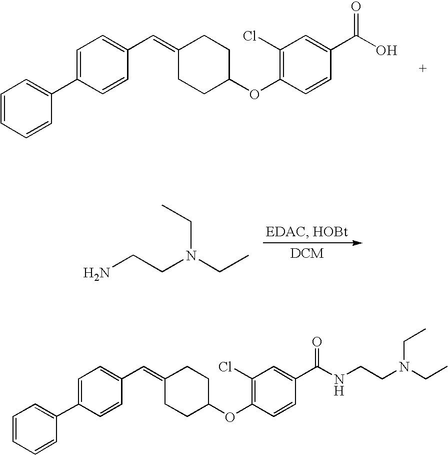

To a mixture of the above acid (110 mg, 0.26 mmol), HOBt.H₂O (53 mg, 0.39 mmol), and EDAC (75 mg, 0.39 mmol) in DCM (10 ml) was added N′,N′-diethylethane-1,2-diamine (0.037 ml, 0.20 mmol) and the mixture was stirred at room temperature under argon for 16 h. The mixture was made alkaline with 1N aqueous NaOH and extracted with DCM. The organic layer was dried (MgSO₄), filtered and concentrated in vacuo. The crude product was purified by HPLC to give 128 mg (88%) of the final product.

### Example 2

### 4-(4-(biphenyl-4-ylmethyl)cyclohexyloxy)-3-chloro-N-(2-diethylamino)ethylbenzamide

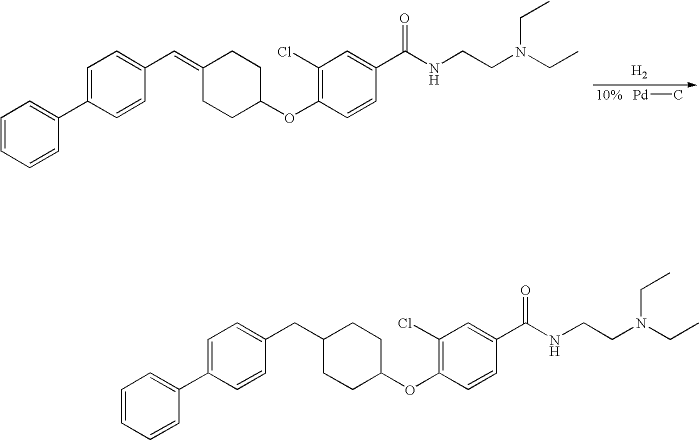

: The compound from the above step (97 mg, 0.18 mmol) was dissolved in MeOH/THF (1:1, 4 ml) and hydrogenated over 10% Pd/C (23.4 mg) at room temperature for 2 h 30 min. The catalyst was removed by filtration, and the filtrate was concentrated in vacuo. The residue was purified by HPLC to give the final product (88 mg, 89%).

### Example 3

### a) tert-Butyl 4-(4-chlorobutanoyl)phenoxy)piperidine-1-carboxylate

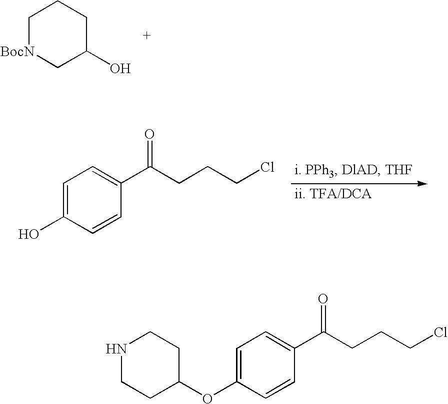

The title compound (9.6 g, 58%) was prepared following the standard Mitsunobu conditions with tert-butyl 4-hydroxypiperidine-1-carboxylate (6.1 g, 30.2 mmol), 4-chloro-1-(4-hydroxyphenyl)butan-1-one (5.0 g, 25.2 mmol), triphenylphosphine (7.9 g, 30.2 mmol) and DIAD (5.9 ml, 30.2 mmol) in THF (150 ml).

### b) 4-(dimethylamino)-1-(4-(piperidin-4-yloxy)phenyl)butan-1-one

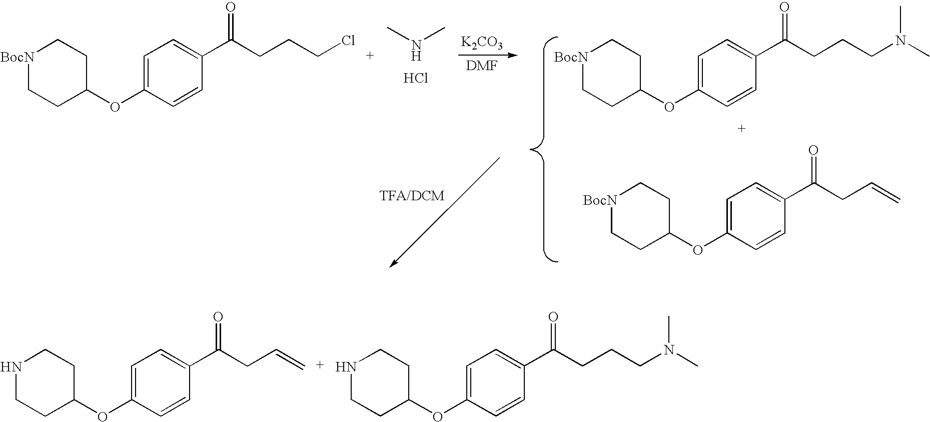

To the (4-chlorobutanoyl)phenoxy) derivative prepared above (1.0 g, 2.6 mmol) in DMF (6 ml) was added excess K₂CO₃ (3 equivalents), and excess dimethylamine hydrochloride (3 equivalents), and the reaction mixture was stirred at 60° C. overnight. Standard aqueous work up and extraction with EtOAc gave a mixture of the expected coupled product and the elimination product. N-boc deprotection of the mixture using a mixture of TFA/DCM (3 ml:15 ml) gave 400 mg of a mixture of 4-(dimethylamino)-1-(4-(piperidin-4-yloxy)phenyl)butan-1-one and the elimination product. This was used as such for the next reductive amination step. The experimental conditions are not optimized.

### c) 4-(Dimethylamino)-1-(4-(1-((9-ethyl-9H-carbazol-3-yl)methyl)piperidin-4-yloxy)phenyl)butan-1-one

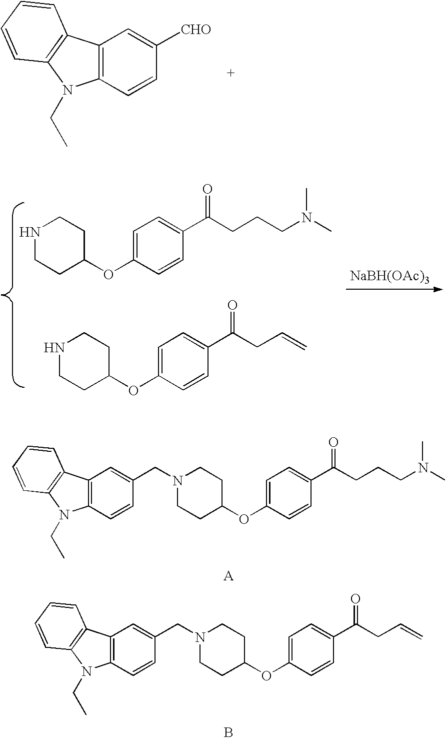

Reductive amination of 9-ethylcarbazole-3-carboxaldehyde (300 mg, 1.34 mmol) and the above prepared mixture (390 mg) with NaBH(OAc)₃ (370 mg, 1.8 mmol) in THF (10 ml) following the standard experimental procedure gave a mixture of two products. The mixture was purified by HPLC methods and obtained the expected product A (168 mg) and the elimination product B (600 mg).

### Example 4

### 5-(Dimethylamino)-2-(4-(1-((9-ethyl-9H-carbazol-3-yl)methyl)piperidin-4-yloxy)phenyl)pentan-2-ol

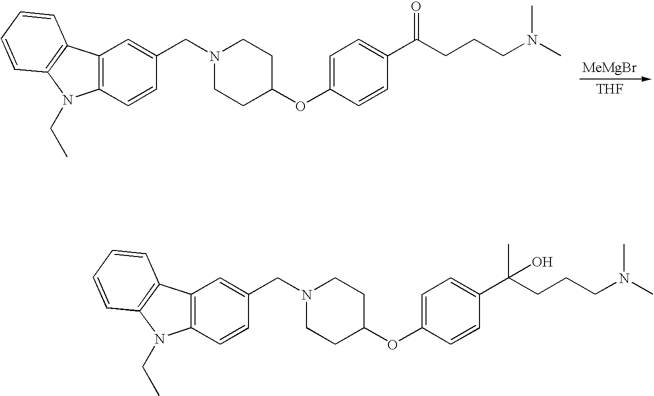

A solution of MeMgBr in THF (3M, 0.2 ml) was added dropwise to a stirred solution of the above prepared phenylbutanone derivative (60 mg, 0.12 mmol) in THF (1 ml) at 0° C. under argon. The reaction mixture was allowed to warm to room temperature and stirred overnight. LC-MS showed 80% conversion to the expected product. After standard aqueous work up with EtOAc, the crude product was purified by HPLC. The experimental conditions are not optimized.

### Example 5

### 4-((1-((9-ethyl-9H-carbazol-3-yl)methyl)piperidin-4-yl)methyl)-N-(3-(2-methylpiperidin-1-yl)propyl)benzamide

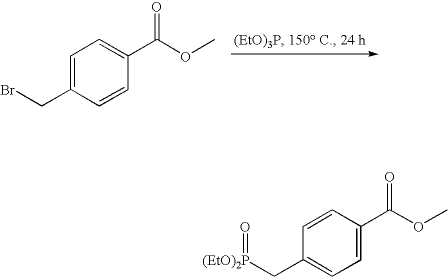

The 4-(bromomethyl)benzoate (25.0 g, 109 mmol) and triethyl phosphite (24.2 mL, 142 mmol) were combined and the mixture was heated at 150° C., and stirred overnight. Excess starting material was removed by distillation at 160° C. to leave the product (23.4 g, 75%) as a colorless oil.

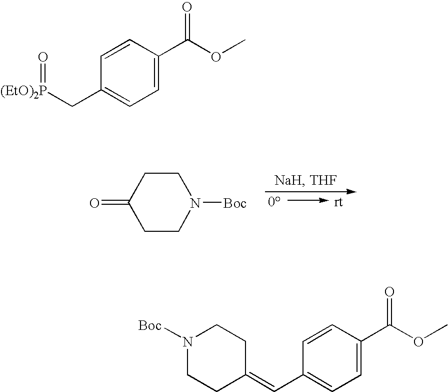

15-crown-5 (1.96 g, 8.9 mmol) and the product from the previous reaction (30.0 g, 105 mmol) were dissolved in THF (150 mL) and cooled to 0° C., and NaH (95% anhydrous; 2.65 g, 105 mmol) was added. The reaction mixture was Stirred for 30 minutes at room temperature, placed in an ice bath then tert-butyl 4-oxopiperidine-1-carboxylate (20.9 g, 105 mmol) in THF (75 mL) was added dropwise over 40 min. The mixture was stirred overnight at room temperature. The mixture was diluted with water (150 mL) and extracted with EtOAc (2×150 mL). The organic layer was washed with saturated aqueous NaHCO₃ (100 mL), saturated NaCl_{aq} (100 mL), dried over anhydrous MgSO₄, filtered, and concentrated in vacuo. The crude product was purified by column chromatography on silica gel (hexane/EtOAc 80:20) to give a mixture of methyl and ethyl esters (27.5 g, 79%) as a semisolid that was used as is.

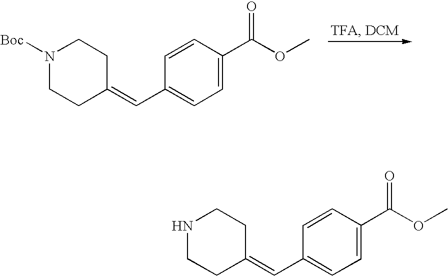

To a solution of ester (10.5 g, 31.7 mmol) in DCM (60 mL), was added TFA (30 mL) and the reaction mixture stirred at room temperature for 4 h. The solvent was removed under vacuum, the oil partitioned between DCM (100 ml) and NaOH (1N, 100 ml). Tand the he aqueous layer was extracted once more with DCM (100 ml). The combined organic layers were dried over sodium sulfate, filtered, and concentrated under vacuum. The final product was obtained as an amber colored oil (7.2 g, 98%).

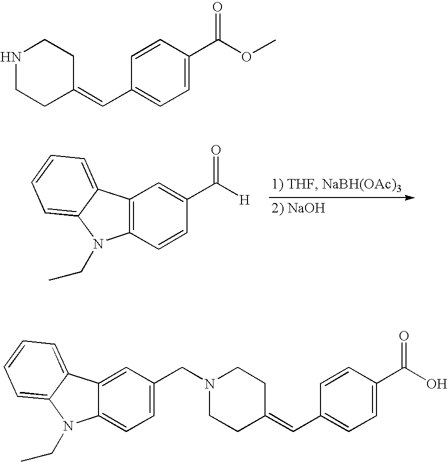

To a solution of 4-piperidin-4-ylidenemethyl-benzoic acid methyl ester (2.00 g, 8.6 mmol) and 9-ethyl-9H-carbazole-3-carbaldhyde (4.82 g, 21.6 mmol) in THF (70 mL), sodium triacetoxyborohydride (4.58 g, 21.6 mmol) was added and the reaction mixture stirred overnight. Methanol (10 ml) and NaOH_{aq} (10N, 12 mL, 120 mmol) were added, and the mixture stirred overnight. The reaction mixture was poured into EtOAc (200 mL), HCl_{aq} (4N, 38 mL, 150 mmol) was added, the layers separated, the organic layer washed with saturated aqueous sodium chloride, dried over anhydrous MgSO₄, filtered and the volume reduced under vacuum until crystals started to form. The precipitate was collected by filtration, washed with EtOAc, and dried in vacuo to give the product (3.01 g, 82%) as a white solid.

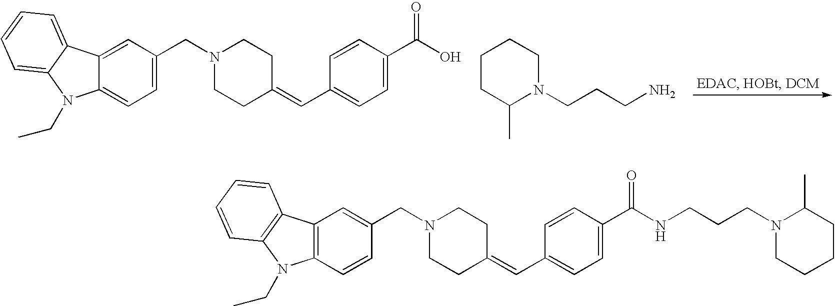

The compound was prepared using a method as previously described. The yield was 83% of the white hydrochloride salt.

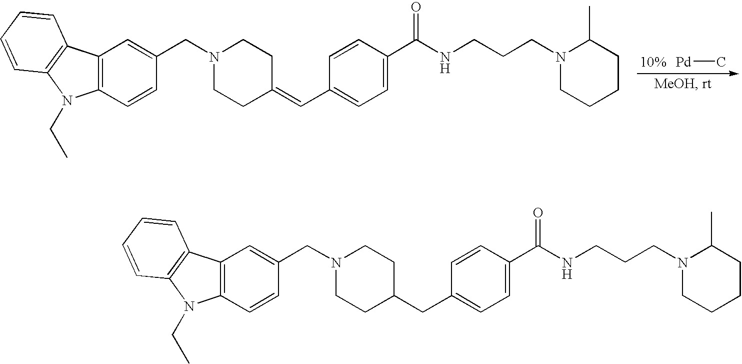

The starting material (130 mg, 0.231 mmol) was dissolved in MeOH (10 ml) and Pd—C (10%, 90 mg) was added. The reaction was stirred overnight under a balloon of hydrogen. The mixture was filtered through filter aid, washed with methanol, reduced under vacuum and purified using preparative HPLC. The final product (95 mg, 73%) was obtained as a white hydrochloride salt.

### Example 6

### 4-(1-(biphenyl-4-ylsulfonyl)piperidin-4-yloxy)-3-chloro-N-(3-(2-methylpiperidin-1-yl)propyl)benzamide

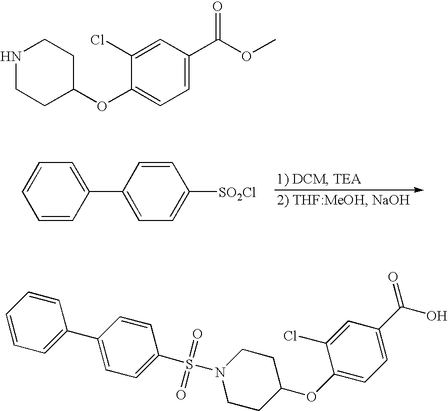

The previously described 3-chloro-4-(piperidin-4-yloxy)-benzoic acid methyl ester (200 mg, 0.74 mmol) was dissolved in DCM (10 mL) and triethylamine (207 μL, 1.48 mmol) and biphenyl-4-sulfonyl chloride (187 mg, 0.74 mmol) were added. The reaction mixture was stirred at room temperature for 30 minutes then the solvent was removed under vacuum. The resulting oil was dissolved in THF (20 mL) and MeOH (5 mL). NaOH (10N, 1.0 mL) was added and the mixture stirred at room temperature overnight. The reaction mixture was poured into EtOAc (100 mL), HCl_{aq} (4N, 5 mL) was added, the layers separated, and the organic layer washed with saturated aqueous sodium chloride, dried over anhydrous MgSO₄, and filtered and the solvent removed under vacuum. The resulting glassy solid (332 mg, 90%) was used without purification:

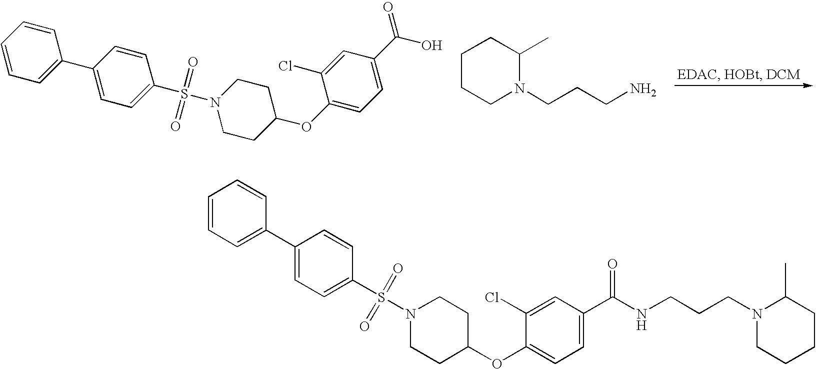

The compound was prepared using a method similar to one previously described. After purification using preparative HPLC and conversion to the hydrochloride salt, the final product was obtained as a white solid (115 mg, 71%).

### Example 7

### N-(4-chloro-3-(trifluoromethyl)phenyl)-4-(4((1-(dimethylamino)cyclohexyl)methylcarbamoyl)benzylidene)piperidine-1-carboxamide

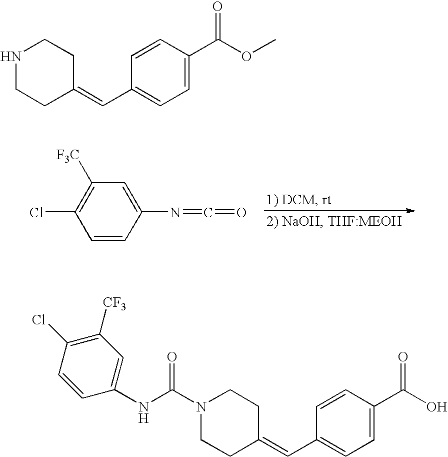

To a solution of 4-piperidin-4-ylidenemethylbenzoic acid methyl ester (192 mg, 0.86 mmol) in DCM (5 mL) was added 1-chloro-4-isocyanato-2-trifluoromethylbenzene (200 mg, 0.86 mmol) at room temperature. After 15 minutes, the solvent was removed under vacuum and THF (20 mL), MeOH (5 mL), and NaOH (10N, 1.0 mL) were added. The mixture was stirred at room temperature overnight. The reaction mixture was poured into EtOAc (100 mL), HCl_{aq} (4N, 5 mL) was added, the layers separated and the organic layer washed with saturated aqueous sodium chloride, and dried over anhydrous MgSO₄, filtered and the solvent removed under vacuum. The resulting solid (375 mg, 96%) was used without purification.

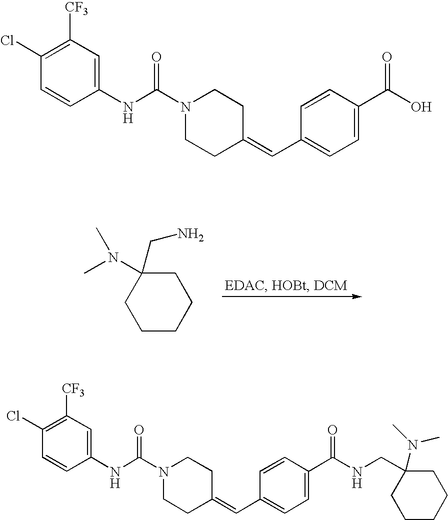

The compound was prepared by a method similar to one previously described. After purification using preparative HPLC and conversion to the hydrochloride salt, the final product was obtained as a white solid (153 mg, 60%).

### Example 8

### 4-(1-(3-chloro-4-fluorophenylcarbamoyl)piperidin-4-yloxy)-N-methylpicolinamide

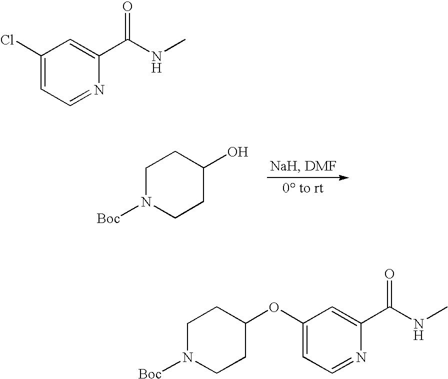

To a mixture of anhydrous sodium hydride (95%, 182 mg, 7.21 mmol) in DMF (anhydrous, 10 mL) cooled to 0° C., was added dropwise 4-hydroxypiperidine-1-carboxylic acid tert-butyl ester (1.45 g, 7.21 mmol) dissolved in DMF (5 mL). The mixture was allowed to warm to room temperature then 4-chloropyridine-2-carboxylic acid methyl ester (1.50 g, 7.21 mmol) dissolved in DMF (10 mL) was added dropwise over 10 min and the mixture was stirred overnight. The mixture was poured into ethyl acetate (100 mL), washed with saturated NaCl (3×50 mL), dried over magnesium sulfate, filtered, and the solvent removed under vacuum. The residue was purified by flash chromatography on silica gel (hexane/EtOAc 50:50) to give the product as colorless oil (2.00 g, 51%).

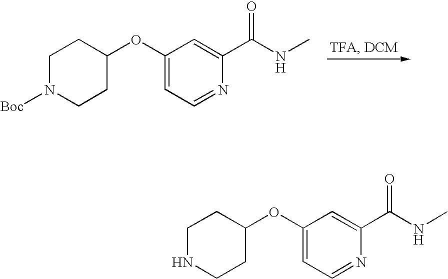

4-(piperidin-4-yloxy)-pyridine-2-carboxylic acid methylamide was prepared using a method similar to one previously described. A quantitative yield of an oil was obtained.

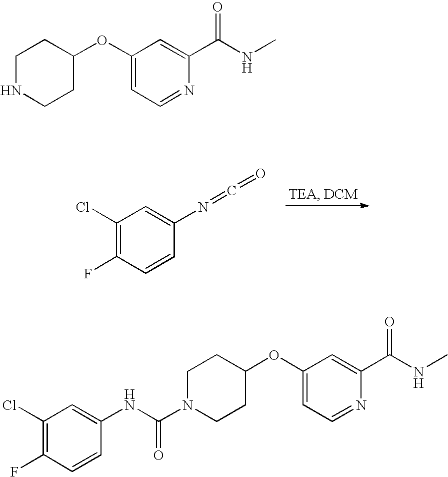

The compound was prepared using a method similar to one previously described. The residue was purified by flash chromatography on silica gel (hexane/EtOAc 50:50) to give the product as white solid (62%).

### Example 9

### N-methyl-4-(1-(4-(phenylethynyl)benzyl)piperidin-4-yloxy)picolinamide

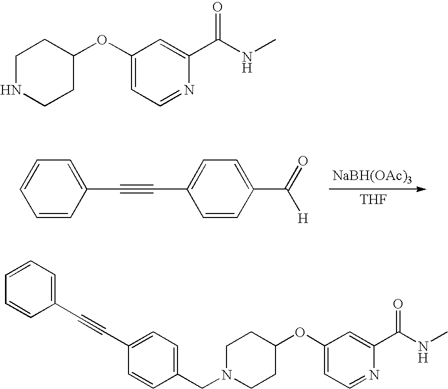

The compound was prepared using a method similar to one previously described. After purification using preparative HPLC and conversion to the hydrochloride salt, the final product was obtained as a white solid (120 mg, 59%).

### Example 10

### N-methyl-4-(1-(naphthalen-1-ylsulfonyl)piperidin-4-yloxy)picolinamide

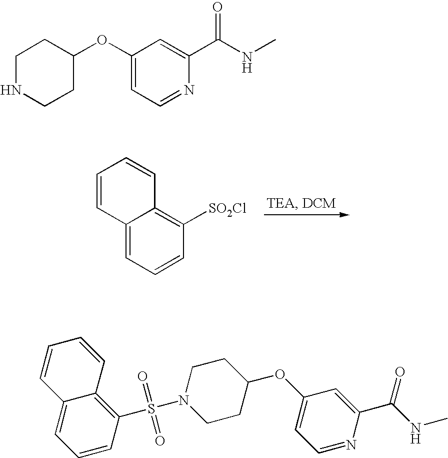

The compound was prepared using a method similar to one previously described. After purification using preparative HPLC, the final product was obtained as a white solid (110 mg, 81%).

Table 1 shows the structure of specific compounds within the invention, wherein ortho, meta and para refer to the position of substituents on the central benzene ring (in other embodiments, the latter may be a pyridine ring), and wherein said structures have one of the indicated formulas:

TABLE 1

| | |
|---|---|
| 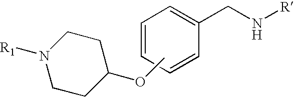 | 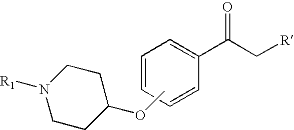 |
| 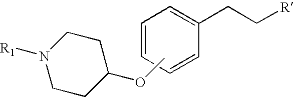 | 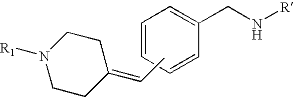 |

| | ortho, meta, | | |
|---|---|---|---|
| Cpd | para | R₁ | R′ |
| 1 | Ortho | 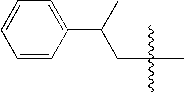 | 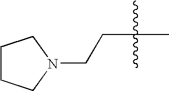 |
| 2 | Ortho | 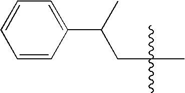 | 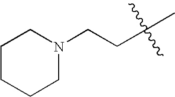 |
| 3 | Ortho | 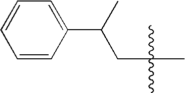 | 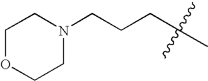 |
| 4 | Ortho | 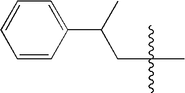 | 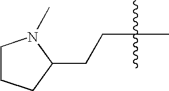 |
| 5 | Ortho | 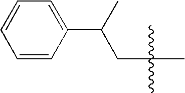 | 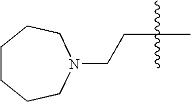 |
| 6 | Ortho | 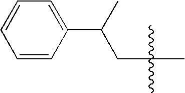 | 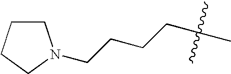 |
| 7 | Ortho | 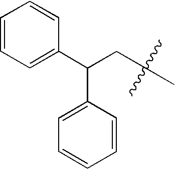 | 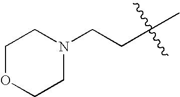 |
| 8 | Ortho | 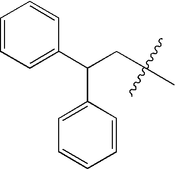 | 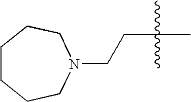 |
| 9 | Ortho | 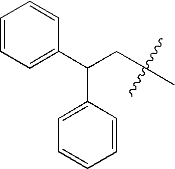 | 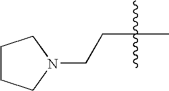 |
| 10 | Ortho | 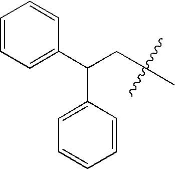 | 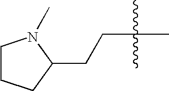 |
| 11 | Ortho | 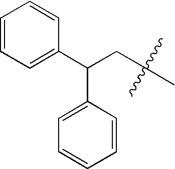 | 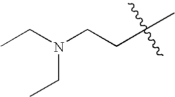 |
| 12 | Ortho | 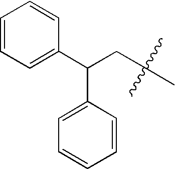 | 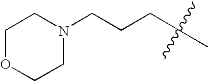 |
| 13 | ortho | 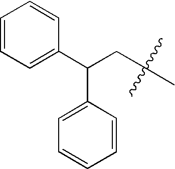 | 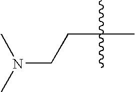 |
| 14 | ortho | 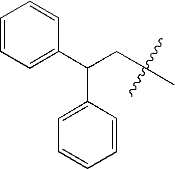 | 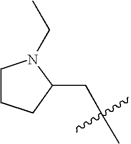 |
| 15 | ortho | 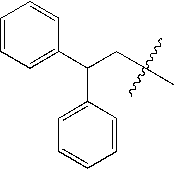 | 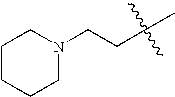 |
| 16 | ortho | 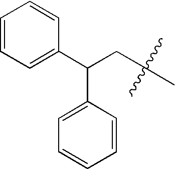 | 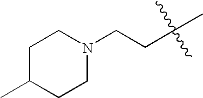 |
| 17 | ortho | 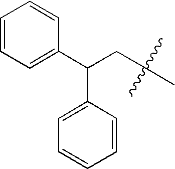 | 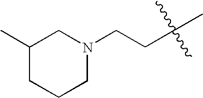 |
| 18 | ortho | 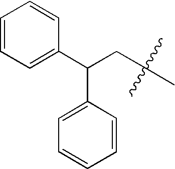 | 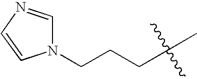 |
| 19 | ortho | 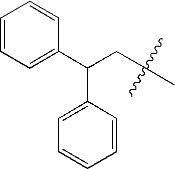 | 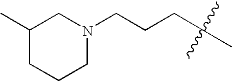 |
| 20 | ortho | 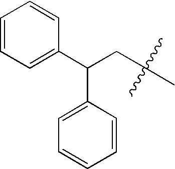 | 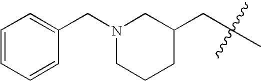 |
| 21 | ortho | 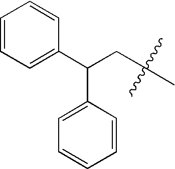 | 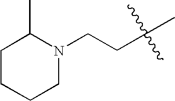 |
| 22 | ortho | 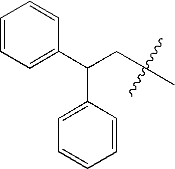 | 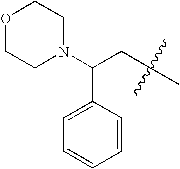 |
| 23 | ortho | 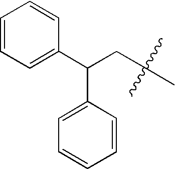 | 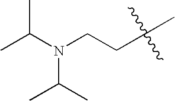 |
| 24 | ortho | 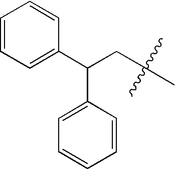 | 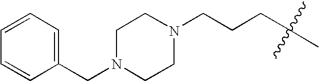 |
| 25 | ortho | 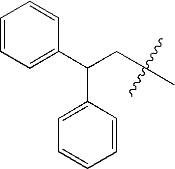 | 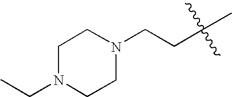 |
| 26 | ortho | 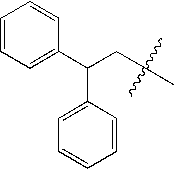 | 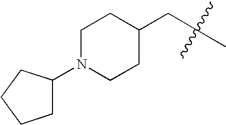 |
| 27 | ortho | 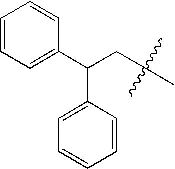 | 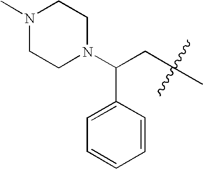 |
| 28 | ortho | 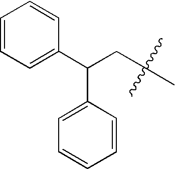 | 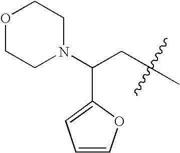 |
| 29 | ortho | 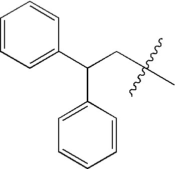 | 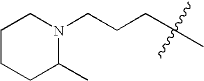 |
| 30 | ortho | 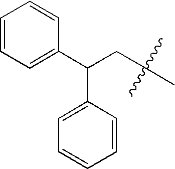 | 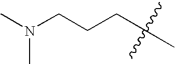 |
| 31 | ortho | 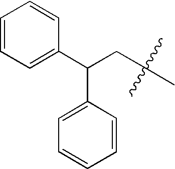 | 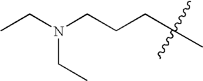 |
| 32 | ortho | 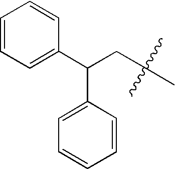 | 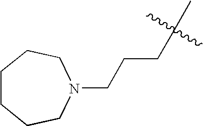 |
| 33 | ortho | 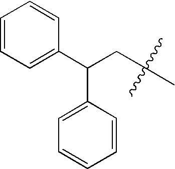 | 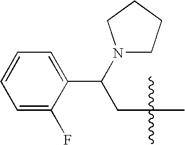 |
| 34 | meta | 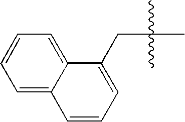 | 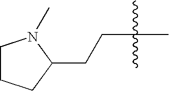 |
| 35 | meta | 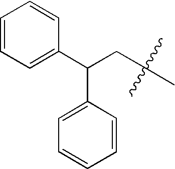 | 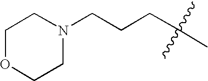 |
| 36 | meta | 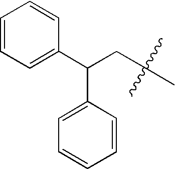 | 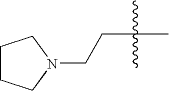 |
| 37 | meta | 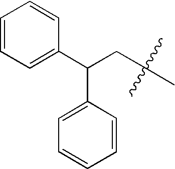 | 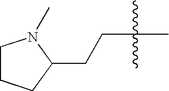 |
| 38 | meta | 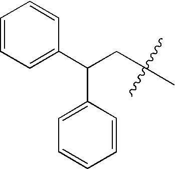 | 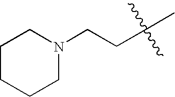 |
| 39 | meta | 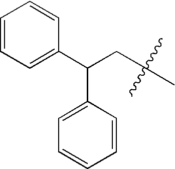 | 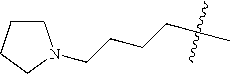 |
| 40 | meta | 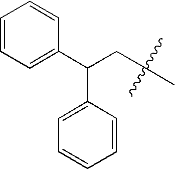 | 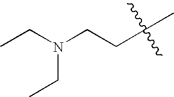 |
| 41 | meta | 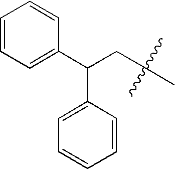 | 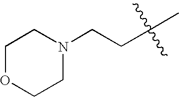 |
| 42 | meta | 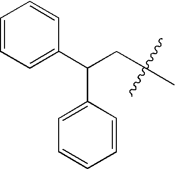 | 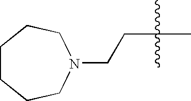 |
| 43 | meta | 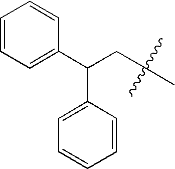 | 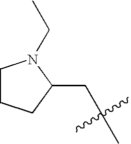 |
| 44 | meta | 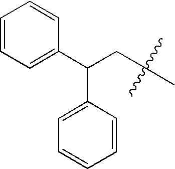 | 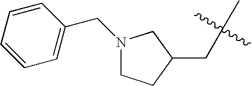 |
| 45 | meta | 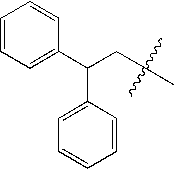 | 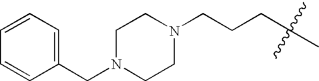 |
| 46 | meta | 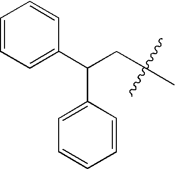 | 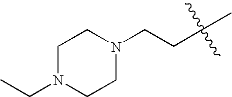 |
| 47 | meta | 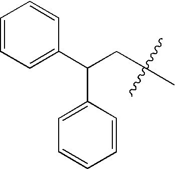 | 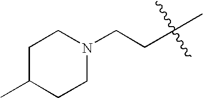 |
| 48 | meta | 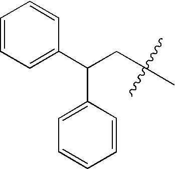 | 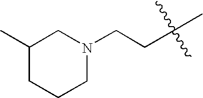 |
| 49 | meta | 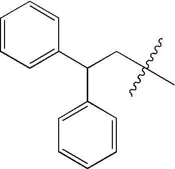 | 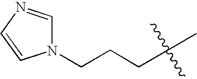 |
| 50 | meta | 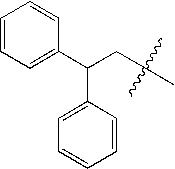 | 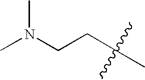 |
| 51 | meta | 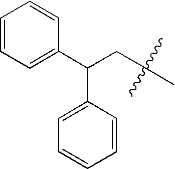 | 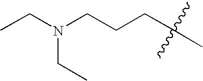 |
| 52 | meta | 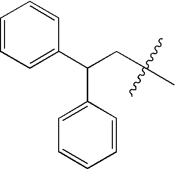 | 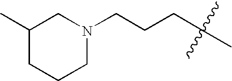 |
| 53 | meta | 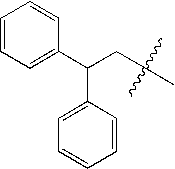 | 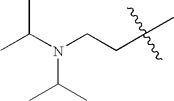 |
| 54 | meta | 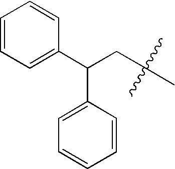 | 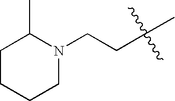 |
| 55 | meta | 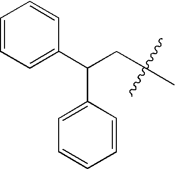 | 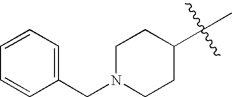 |
| 56 | meta | 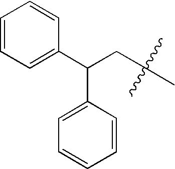 | 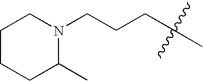 |
| 57 | meta | 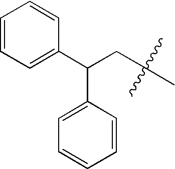 | 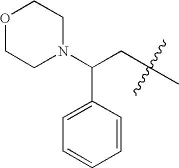 |
| 58 | meta | 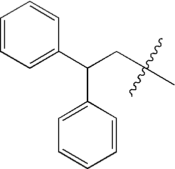 | 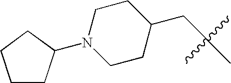 |
| 59 | meta | 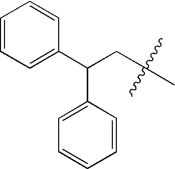 | 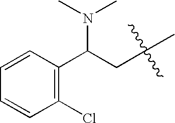 |
| 60 | meta | 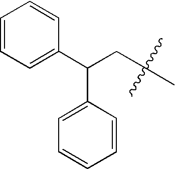 | 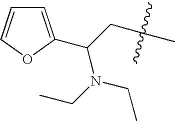 |
| 61 | meta | 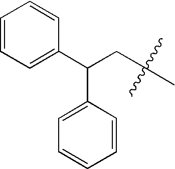 | 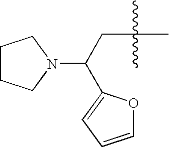 |
| 62 | meta | 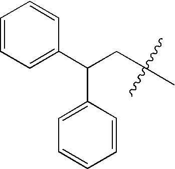 | 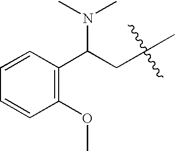 |
| 63 | meta | 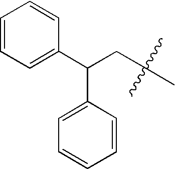 | 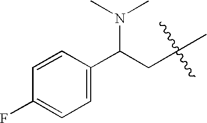 |
| 64 | meta | 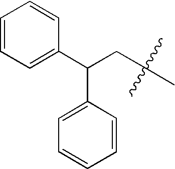 | 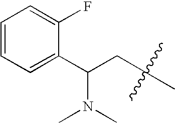 |
| 65 | meta | 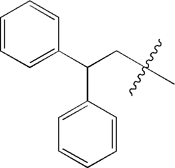 | 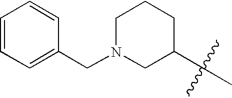 |
| 66 | meta | 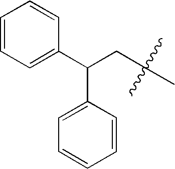 | 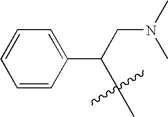 |
| 67 | meta | 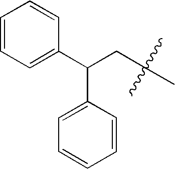 | 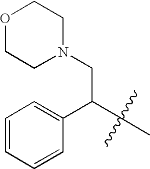 |
| 68 | meta | 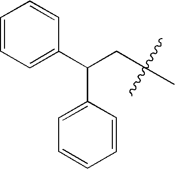 | 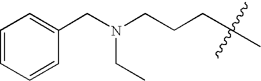 |
| 69 | meta | 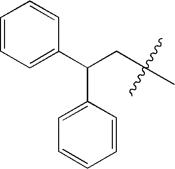 | 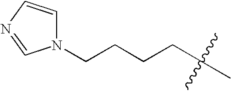 |
| 70 | meta | 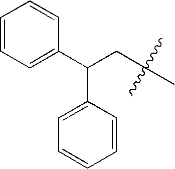 | 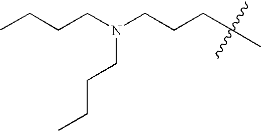 |
| 71 | para | 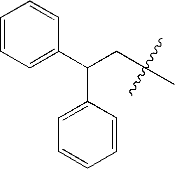 | 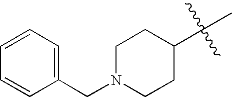 |
| 72 | para | 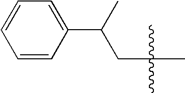 | 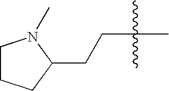 |
| 73 | para | 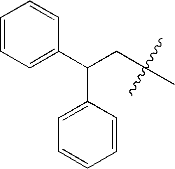 | 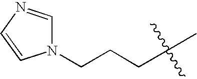 |
| 74 | para | 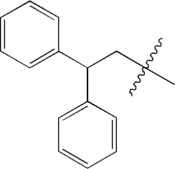 | 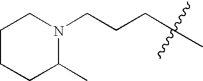 |
| 75 | para | 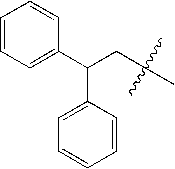 | 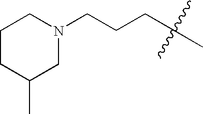 |
| 76 | para | 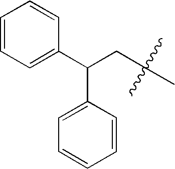 | 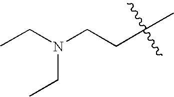 |
| 77 | para | 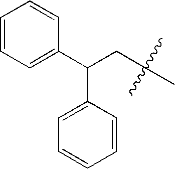 | 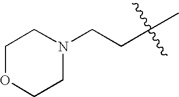 |
| 78 | para | 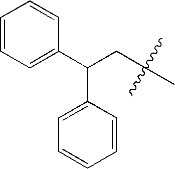 | 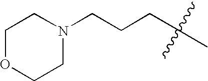 |
| 79 | para | 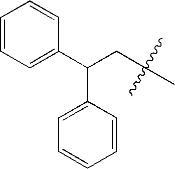 | 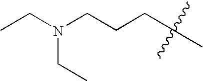 |
| 80 | para | 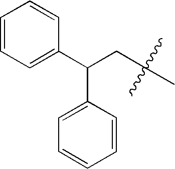 | 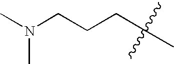 |
| 81 | para | 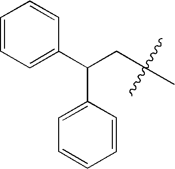 | 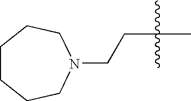 |
| 82 | para | 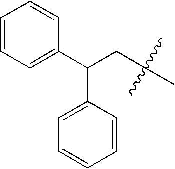 | 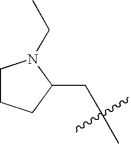 |
| 83 | para | 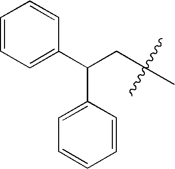 | 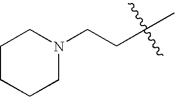 |
| 84 | para | 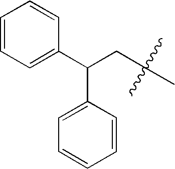 | 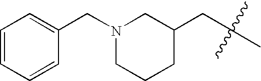 |
| 85 | para | 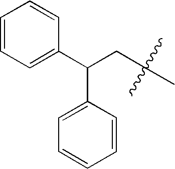 | 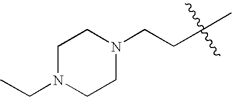 |
| 86 | para | 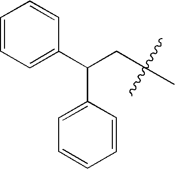 | 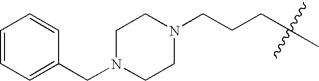 |
| 87 | para | 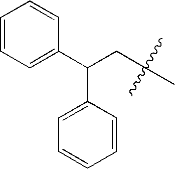 | 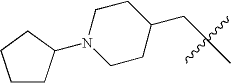 |
| 88 | para | 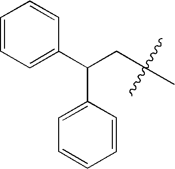 | 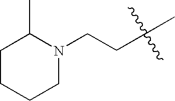 |
| 89 | para | 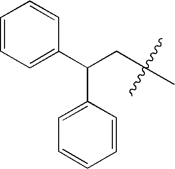 | 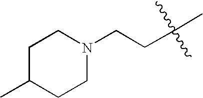 |
| 90 | para | 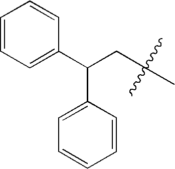 | 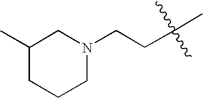 |
| 91 | para | 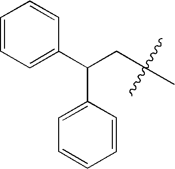 | 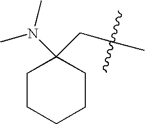 |
| 92 | para | 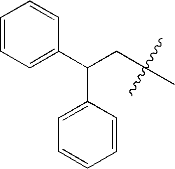 | 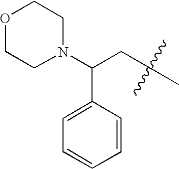 |
| 93 | para | 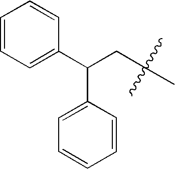 | 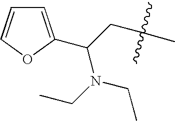 |
| 94 | para | 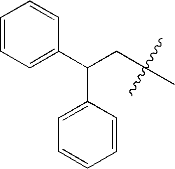 | 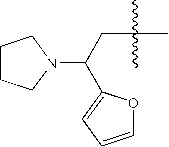 |
| 95 | para | 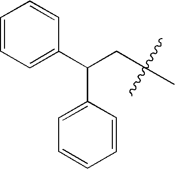 | 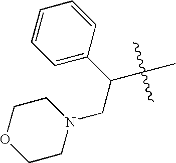 |
| 96 | para | 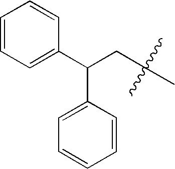 | 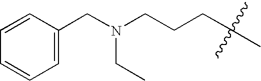 |
| 97 | para | 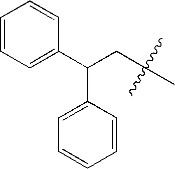 | 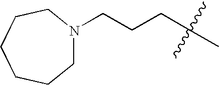 |
| 98 | para | 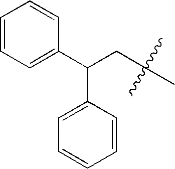 | 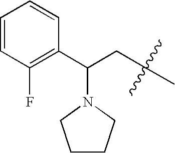 |
| 99 | para | 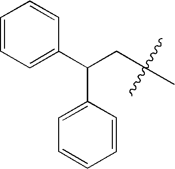 | 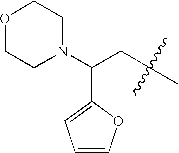 |
| 100 | para | 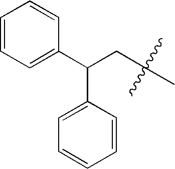 | 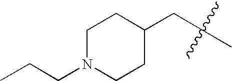 |
| 101 | para | 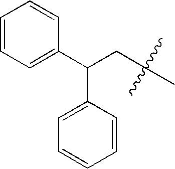 | 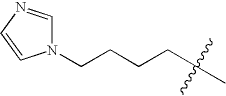 |
| 102 | para | 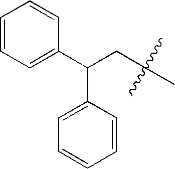 | 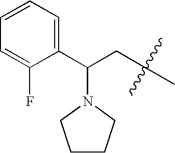 |
| 103 | para | 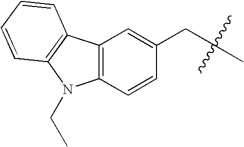 | 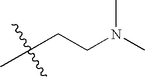 |
| 104 | para | 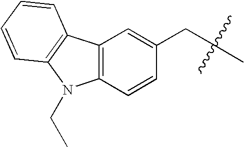 | 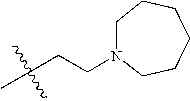 |
| 105 | para | 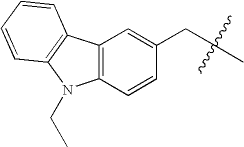 | 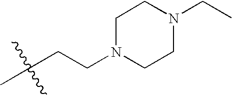 |
| 106 | para | 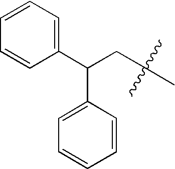 | 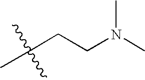 |
| 107 | para | 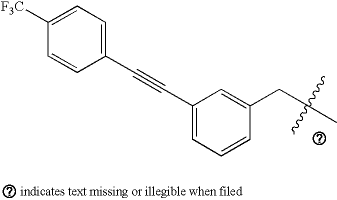 | 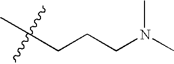 |

Table 2 shows the structure of specific compounds within the invention, wherein ortho, meta and para refer to the position of substituents on the central benzene ring (in other embodiments, the latter may be a pyridine ring), and wherein said structures have one of the indicated 4 formulas:

TABLE 2

| | |
|---|---|
| 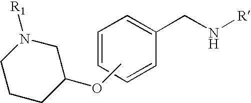 | 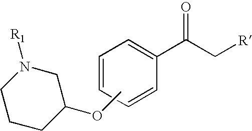 |
| 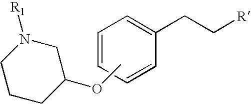 | 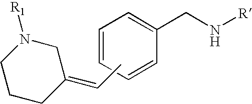 |

| | ortho, | | |
|---|---|---|---|
| | meta, para | R₁ | R′ |
| 1 | meta | 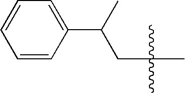 | 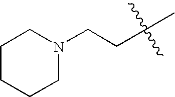 |
| 2 | meta | 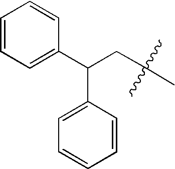 | 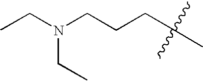 |
| 3 | meta | 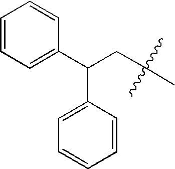 | 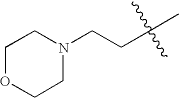 |
| 4 | meta | 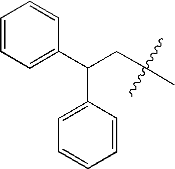 | 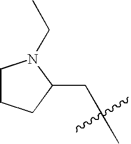 |
| 5 | meta | 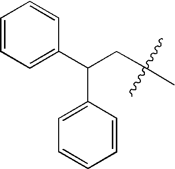 | 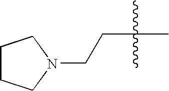 |
| 6 | meta | 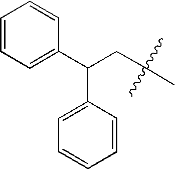 | 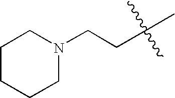 |
| 7 | meta | 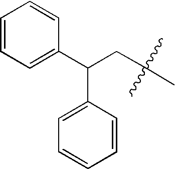 | 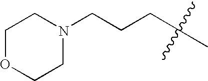 |
| 8 | meta | 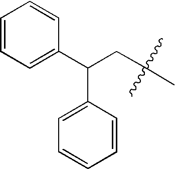 | 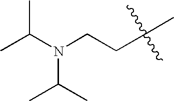 |
| 9 | meta | 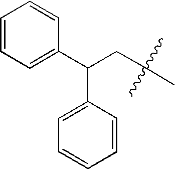 | 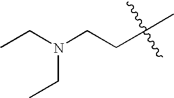 |
| 10 | meta | 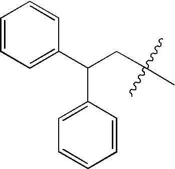 | 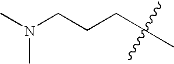 |
| 11 | meta | 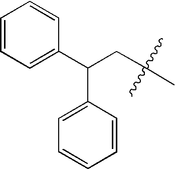 | 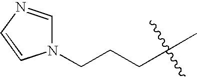 |
| 12 | meta | 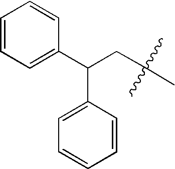 | 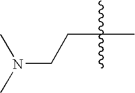 |
| 13 | meta | 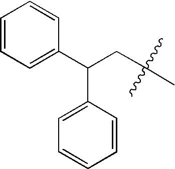 | 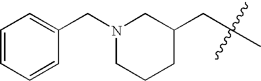 |
| 14 | meta | 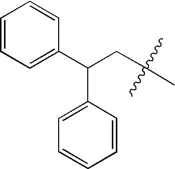 | 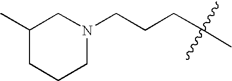 |
| 15 | meta | 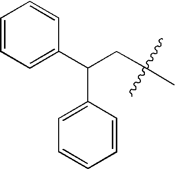 | 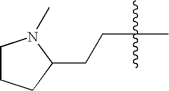 |
| 16 | meta | 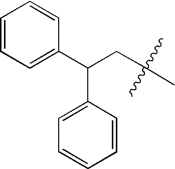 | 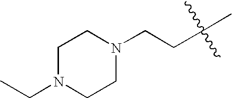 |
| 17 | meta | 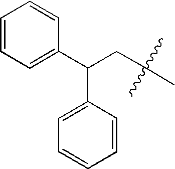 | 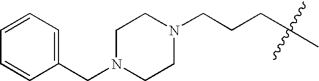 |
| 18 | meta | 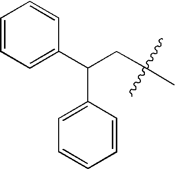 | 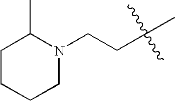 |
| 19 | meta | 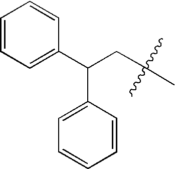 | 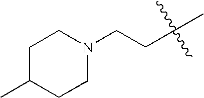 |
| 20 | meta | 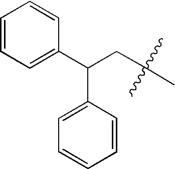 | 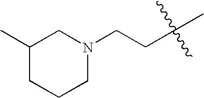 |
| 21 | meta | 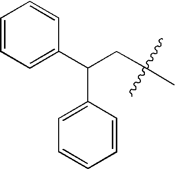 | 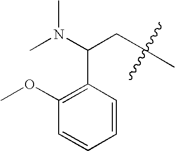 |
| 22 | meta | 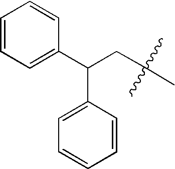 | 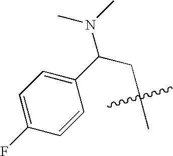 |
| 23 | meta | 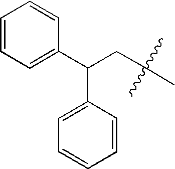 | 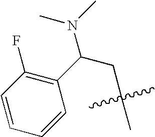 |
| 24 | meta | 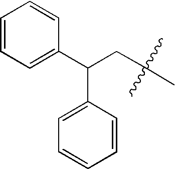 | 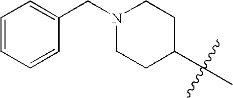 |
| 25 | meta | 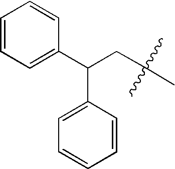 | 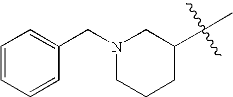 |
| 26 | meta | 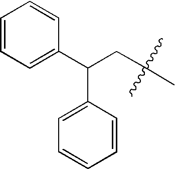 | 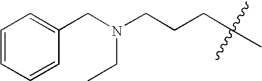 |
| 27 | meta | 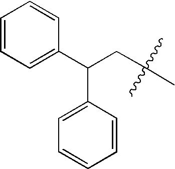 | 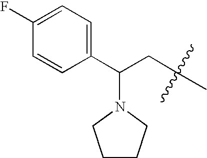 |
| 28 | meta | 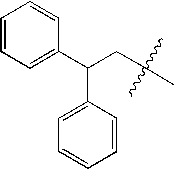 | 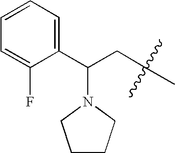 |
| 29 | meta | 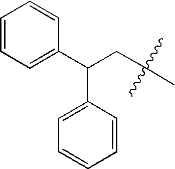 | 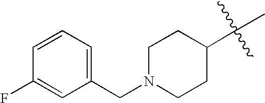 |
| 30 | meta | 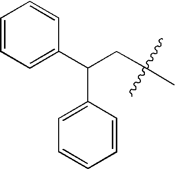 | 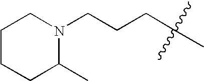 |
| 31 | meta | 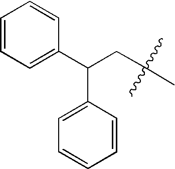 | 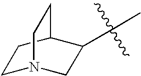 |
| 32 | meta | 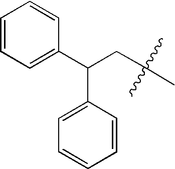 | 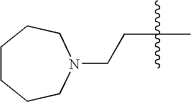 |
| 33 | meta | 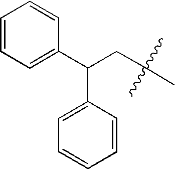 | 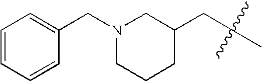 |
| 34 | meta | 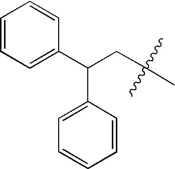 | 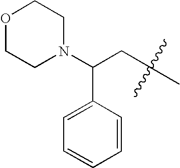 |
| 35 | meta | 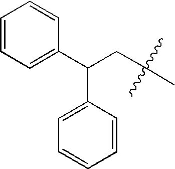 | 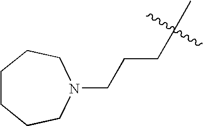 |
| 36 | meta | 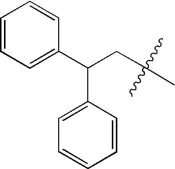 | 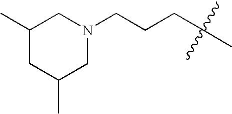 |
| 37 | meta | 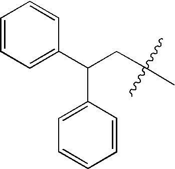 | 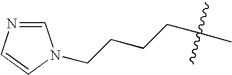 |
| 38 | meta | 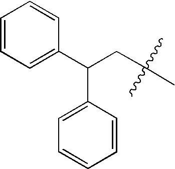 | 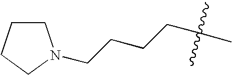 |
| 39 | meta | 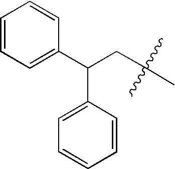 | 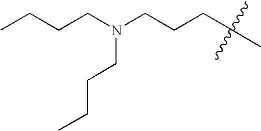 |
| 40 | meta | 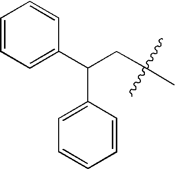 | 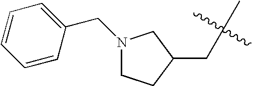 |
| 41 | meta | 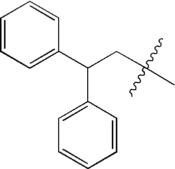 | 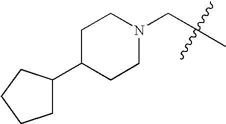 |
| 42 | meta | 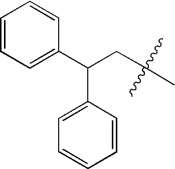 | 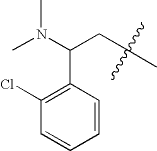 |
| 43 | meta | 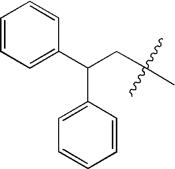 | 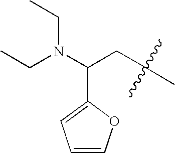 |
| 44 | meta | 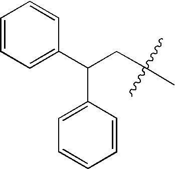 | 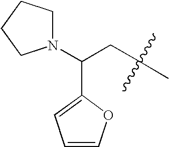 |
| 45 | meta | 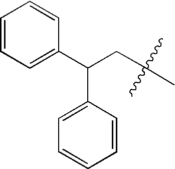 | 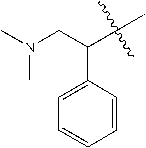 |
| 46 | meta | 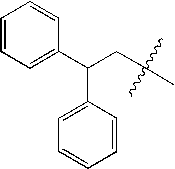 | 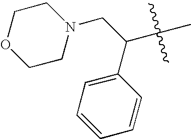 |
| 47 | meta | 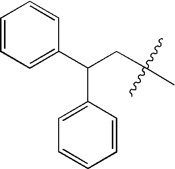 | 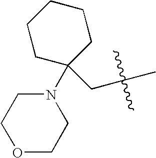 |
| 48 | meta | 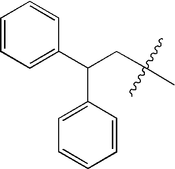 | 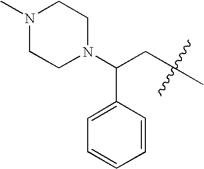 |
| 49 | meta | 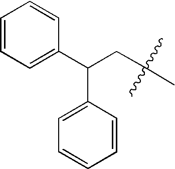 | 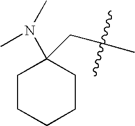 |
| 50 | meta | 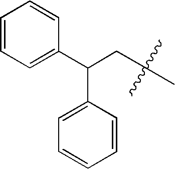 | 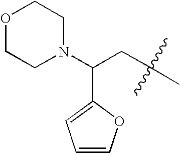 |
| 51 | meta | 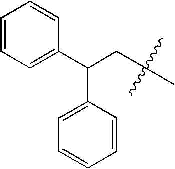 | 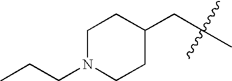 |
| 52 | meta | 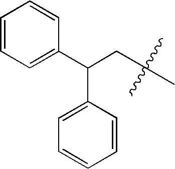 | 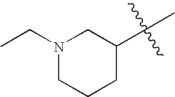 |

Table 3 shows the structure of specific compounds within the invention, wherein ortho, meta and para refer to the position of substituents on the central benzene ring (in other embodiments, the latter may be a pyridine ring), and wherein said structures have one of the indicated 4 formulas:

TABLE 3

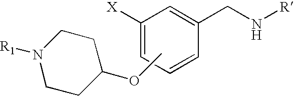

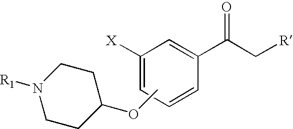

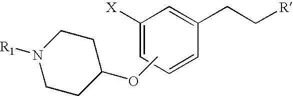

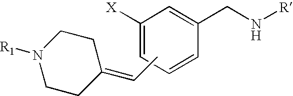

| | X | R₁ | R′ |
|---|---|---|---|
| 1 | OMe | 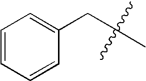 | 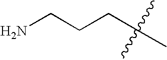 |
| 2 | OMe | 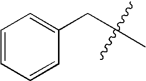 | 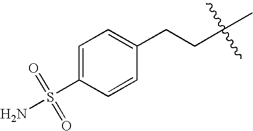 |
| 3 | OMe | 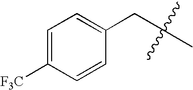 | 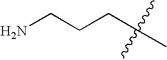 |
| 4 | OMe | 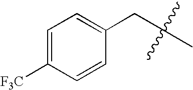 | 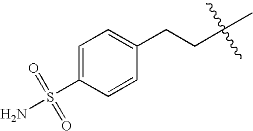 |
| 5 | OMe | 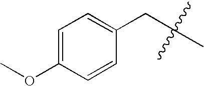 | 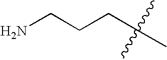 |
| 6 | OMe | 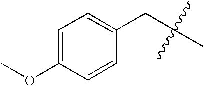 | 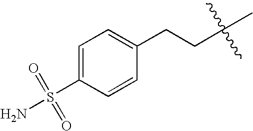 |
| 7 | OMe | 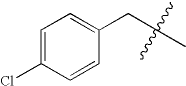 | 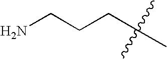 |
| 8 | OMe | 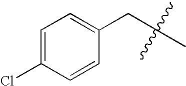 | 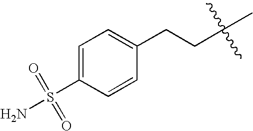 |
| 9 | OMe | 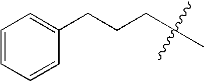 | 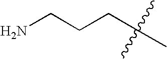 |
| 10 | OMe | 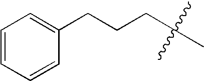 | 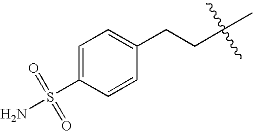 |
| 11 | OMe | 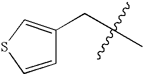 | 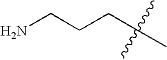 |
| 12 | OMe | 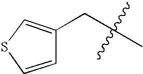 | 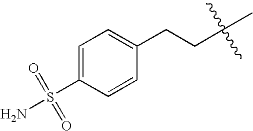 |
| 13 | OMe | 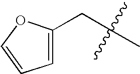 | 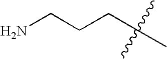 |
| 14 | OMe | 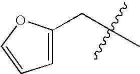 | 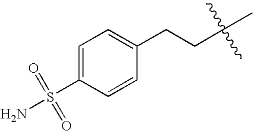 |
| 15 | OMe | 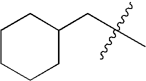 | 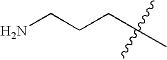 |
| 16 | OMe | 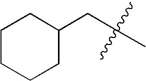 | 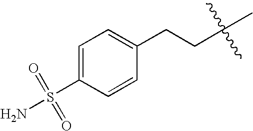 |
| 17 | Cl | 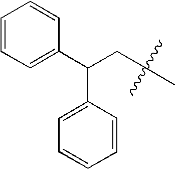 | 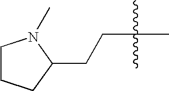 |
| 18 | Cl | 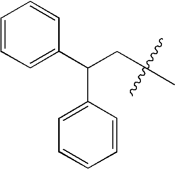 | 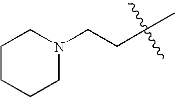 |
| 19 | Cl | 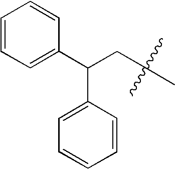 | 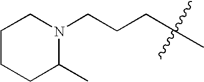 |
| 20 | Cl | 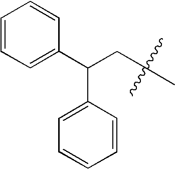 | 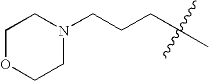 |
| 21 | Cl | 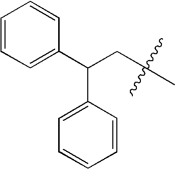 | 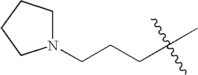 |
| 22 | Cl | 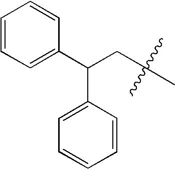 | 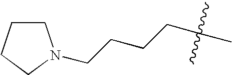 |
| 23 | Cl | 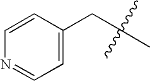 | 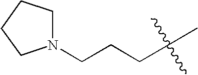 |
| 24 | Cl | 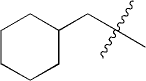 | 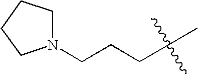 |
| 25 | Cl | 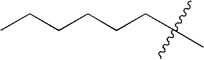 | 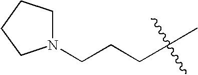 |
| 26 | Cl | 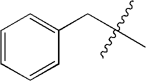 | 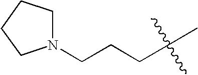 |
| 27 | Cl | 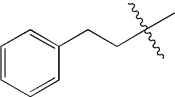 | 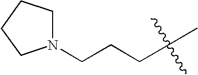 |
| 28 | Cl | 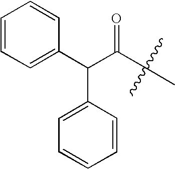 | 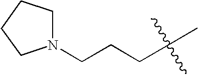 |
| 29 | Cl | 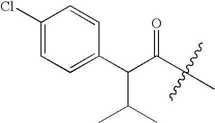 | 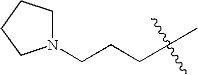 |
| 30 | Cl | 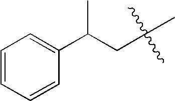 | 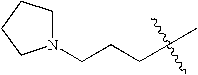 |
| 31 | Cl | 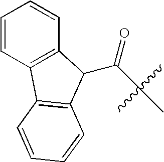 | 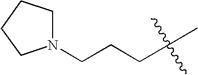 |
| 32 | Cl | 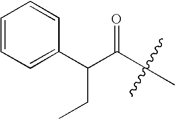 | 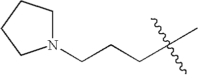 |
| 33 | Cl | 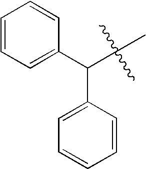 | 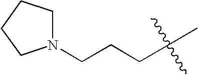 |
| 34 | Cl | 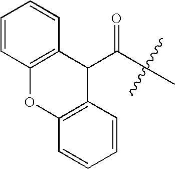 | 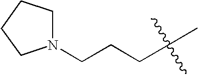 |
| 35 | Cl | 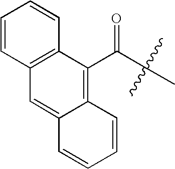 | 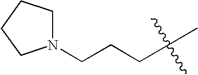 |
| 36 | Cl | 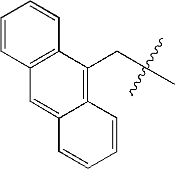 | 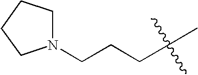 |
| 37 | Cl | 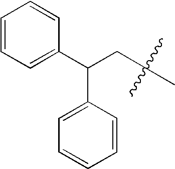 | 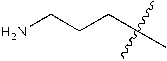 |
| 38 | Cl | 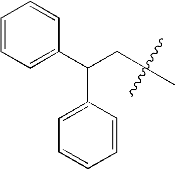 | 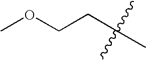 |
| 39 | Cl | 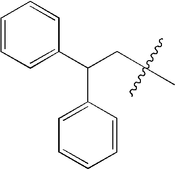 | 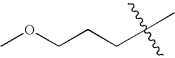 |
| 40 | Cl | 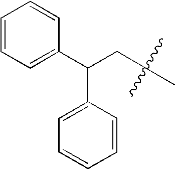 | 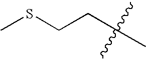 |
| 41 | Cl | 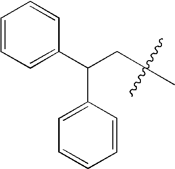 | 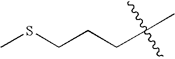 |
| 42 | Cl | 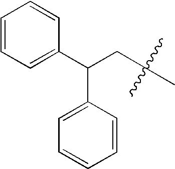 | 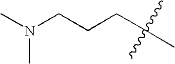 |
| 43 | Cl | 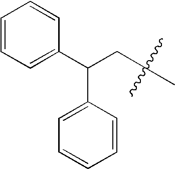 | 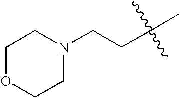 |
| 44 | Cl | 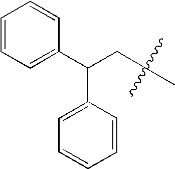 | 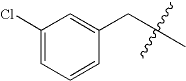 |
| 45 | Cl | 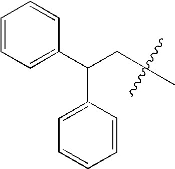 | 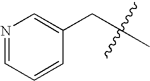 |
| 46 | Cl | 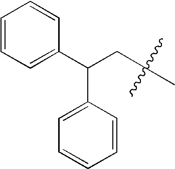 | 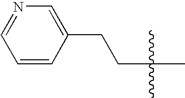 |
| 47 | Cl | 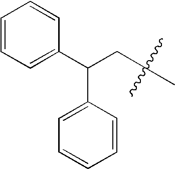 | 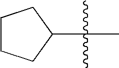 |
| 48 | Cl | 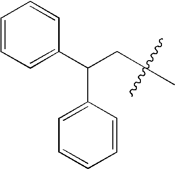 | 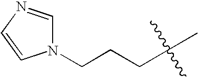 |
| 49 | Cl | 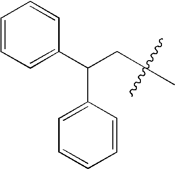 | 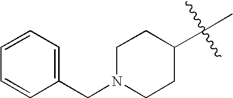 |
| 50 | Cl | 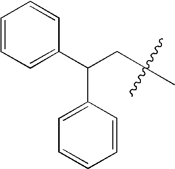 | 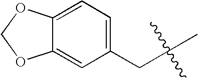 |
| 51 | Cl | 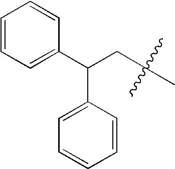 | 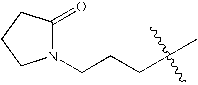 |

TABLE 4

| | |
|---|---|
| 1 | 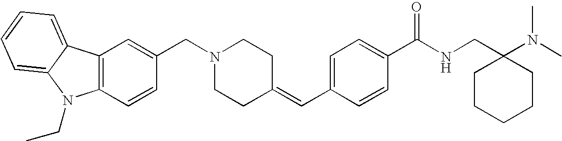 |
| 2 | 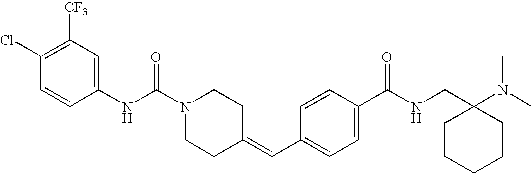 |
| 3 | 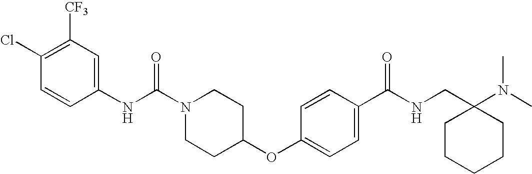 |
| 4 | 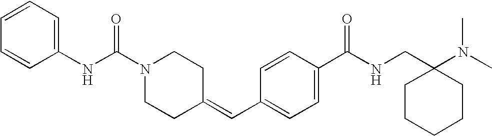 |
| 5 | 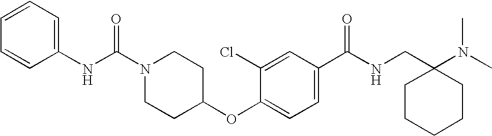 |
| 6 | 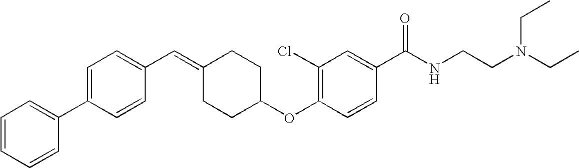 |
| 7 | 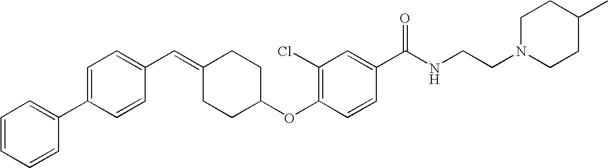 |
| 8 | 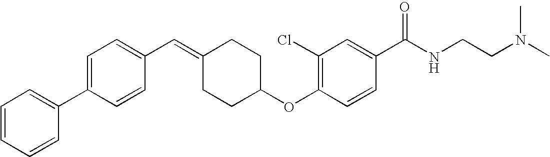 |
| 9 | 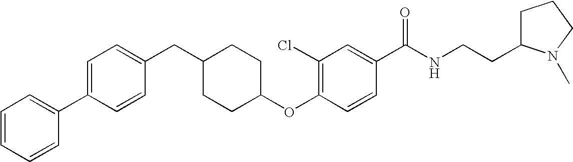 |
| 10 | 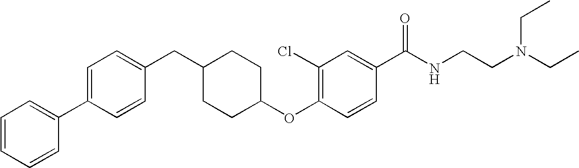 |
| 11 | 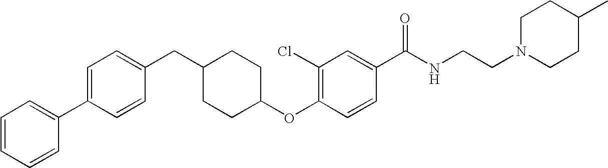 |
| 12 | 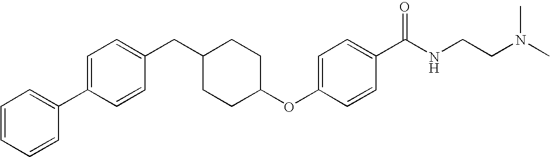 |
| 13 | 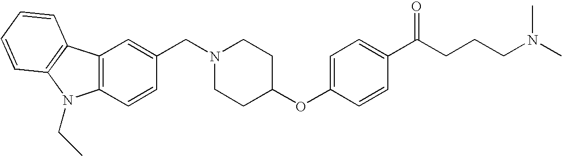 |
| 14 | 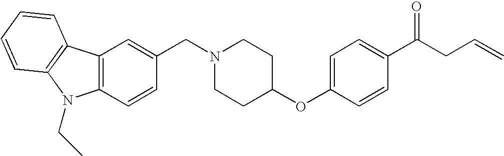 |
| 15 | 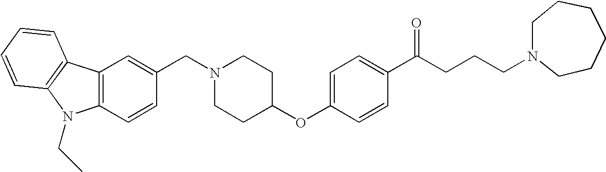 |
| 16 | 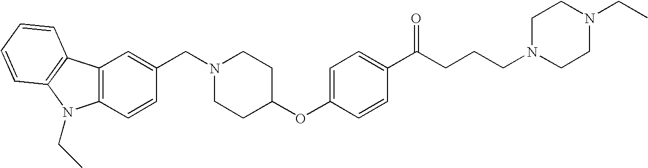 |
| 17 | 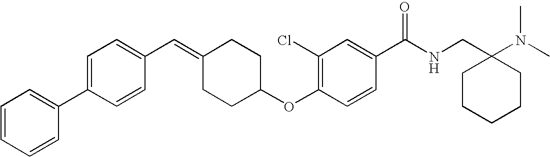 |
| 18 | 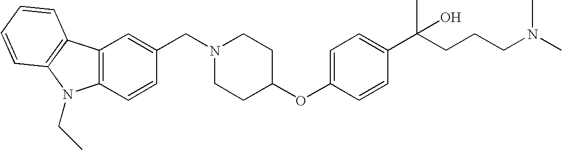 |
| 19 | 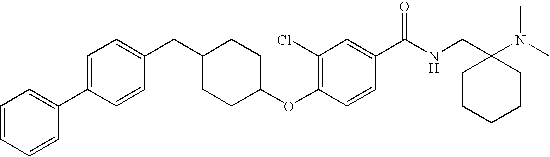 |
| 20 | 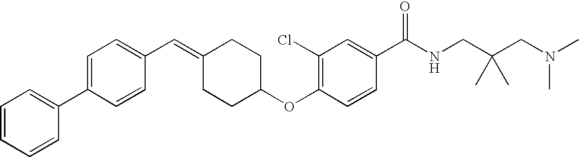 |
| 21 | 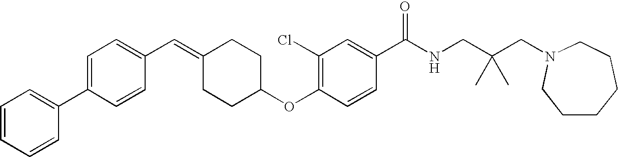 |
| 22 | 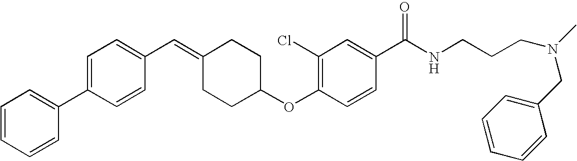 |
| 23 | 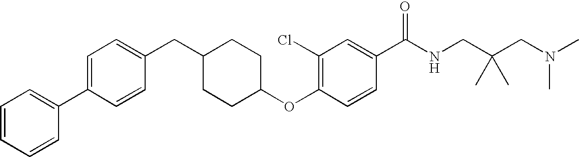 |
| 24 | 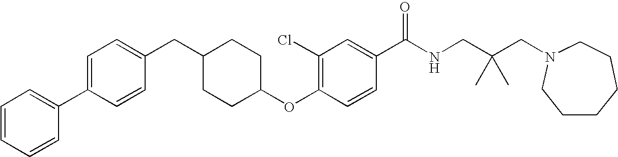 |
| 25 | 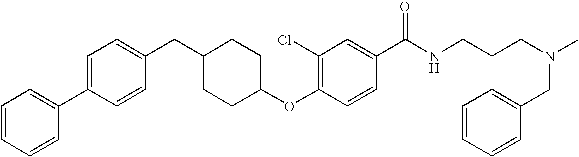 |
| 26 | 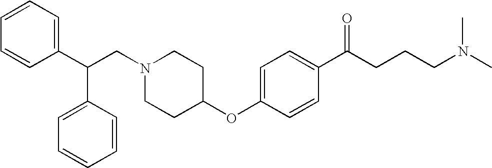 |
| 27 | 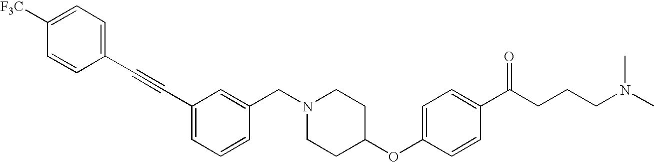 |
| 28 | 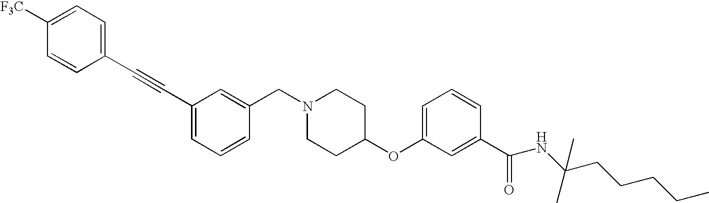 |
| 29 | 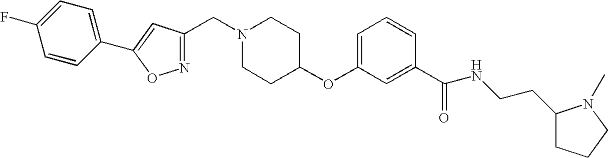 |
| 30 | 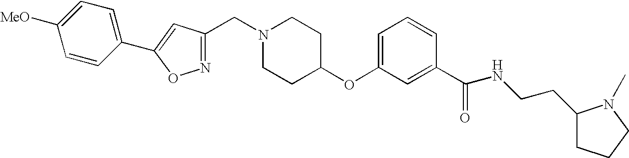 |
| 31 | 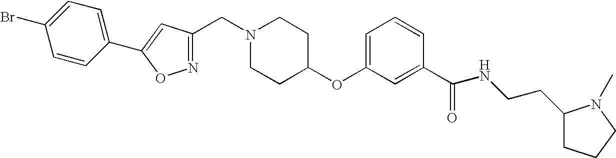 |
| 32 | 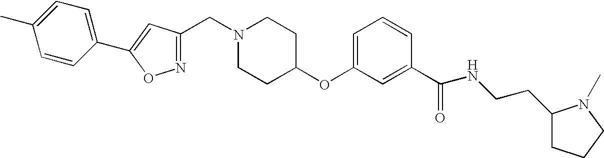 |
| 33 | 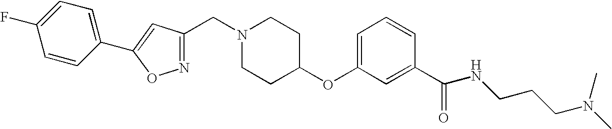 |
| 34 | 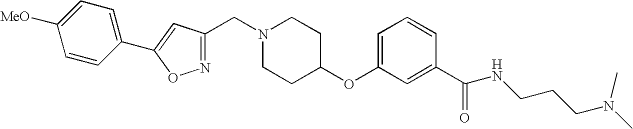 |
| 35 | 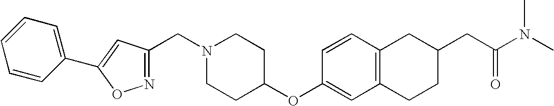 |
| 36 | 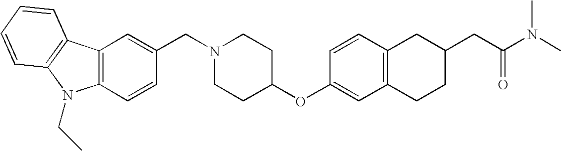 |
| 37 | 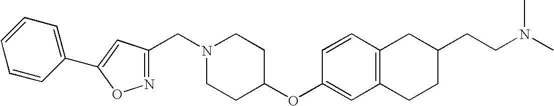 |
| 38 | 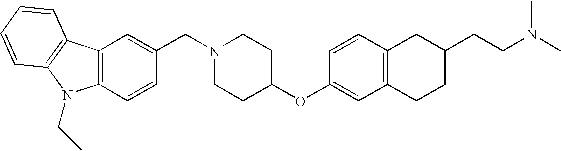 |
| 39 | 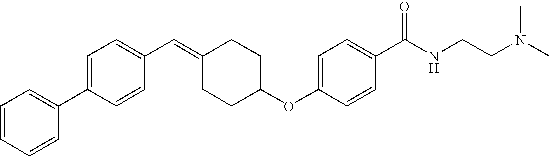 |
| 40 | 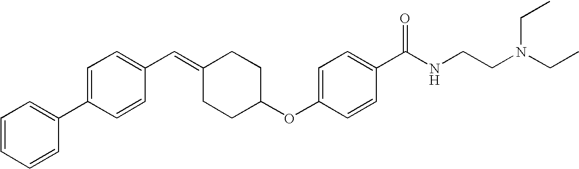 |
| 41 | 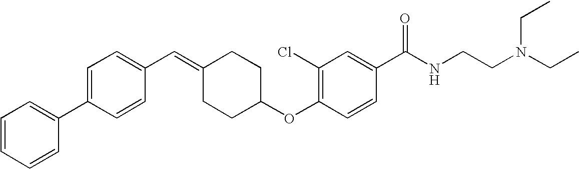 |

TABLE 5

| | |
|---|---|
| 1 | 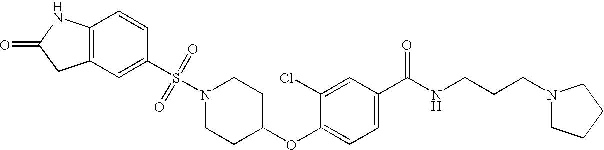 |
| 2 | 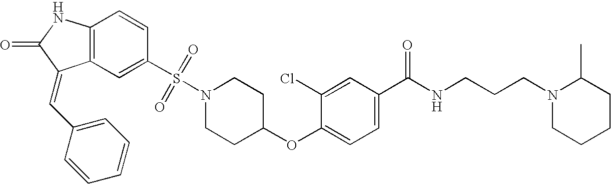 |
| 3 | 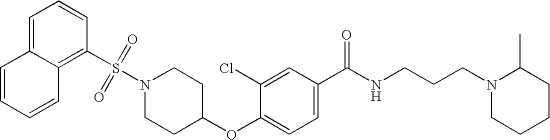 |
| 4 | 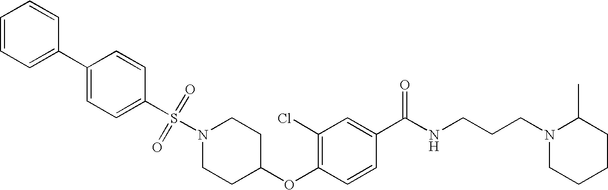 |
| 5 | 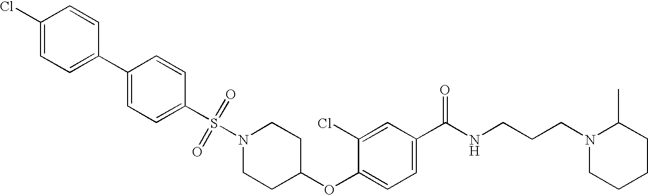 |
| 6 | 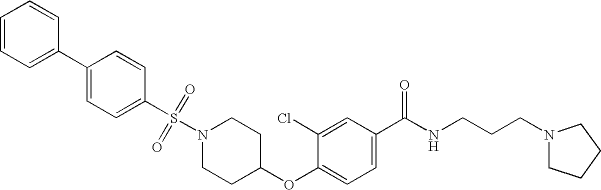 |
| 7 | 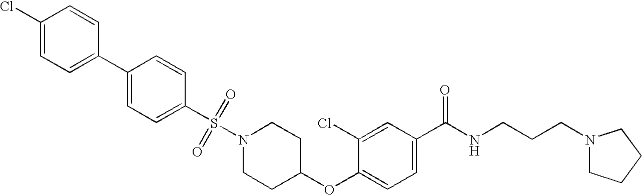 |
| 8 | 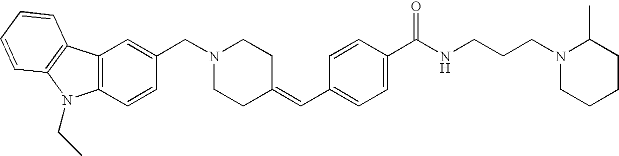 |
| 9 | 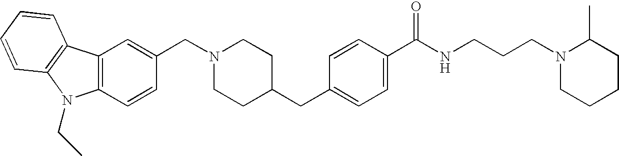 |
| 10 | 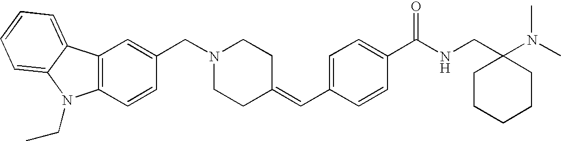 |
| 11 | 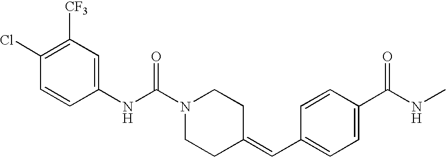 |
| 12 | 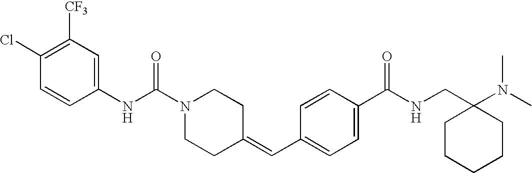 |
| 13 | 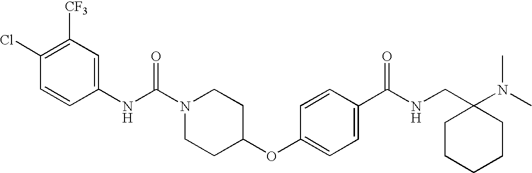 |
| 14 | 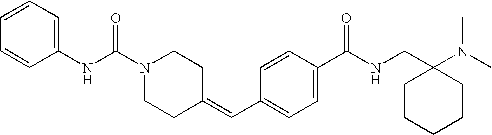 |
| 15 | 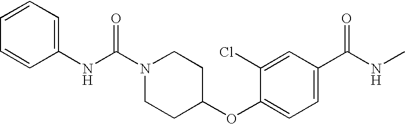 |
| 16 | 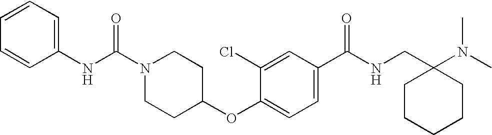 |
| 17 | 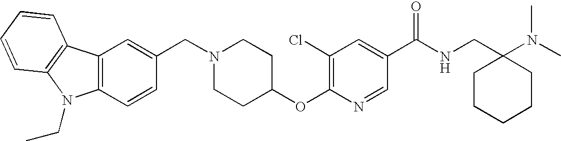 |
| 18 | 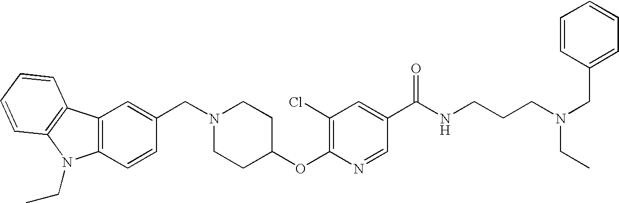 |
| 19 | 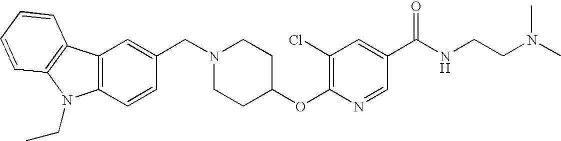 |
| 20 | 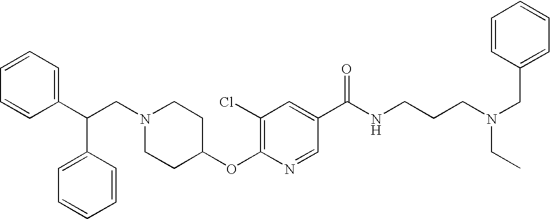 |
| 21 | 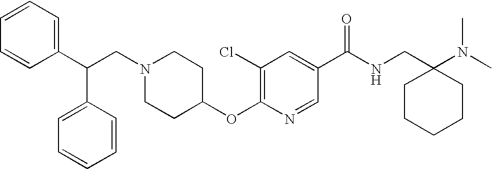 |

In addition, it is to be appreciated that one optical isomer may have favorable properties over the other and thus the disclosure herein may include either optically active isomer if that isomer has advantageous physiological activity in accordance with the methods of the invention. Unless stated otherwise, the disclosure of an optically active isomer herein is intended to include all enantiomers or diastereomers of said compound so long as said structure has the activity described herein for the class of compounds of which said structure is a member.

In other preferred embodiments, the compounds of the invention include the following specific structures:

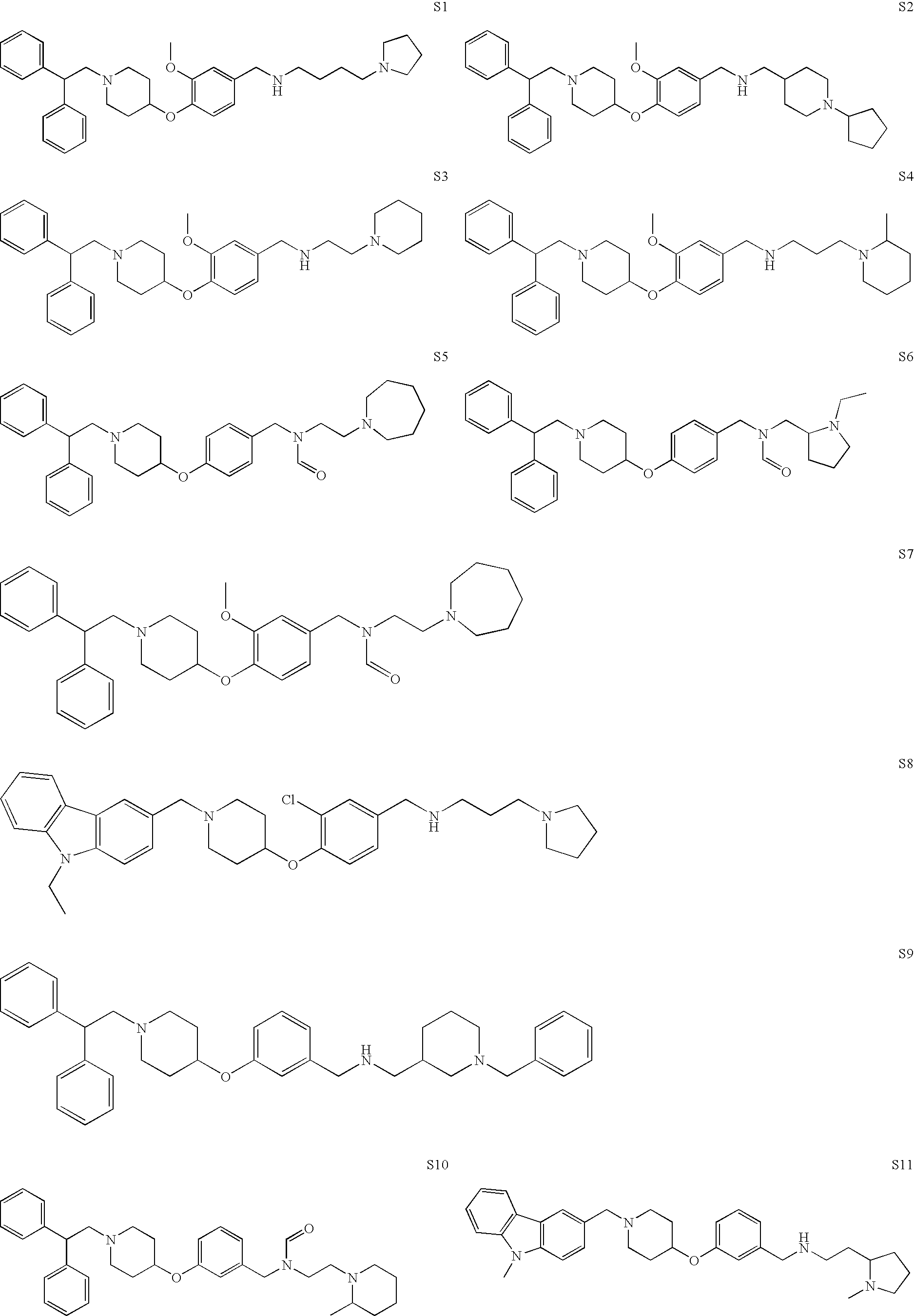

TABLE 6

| Gene | Gene | |
|---|---|---|
| No. | Identifier | Gene Name |
| 1 | NM_004624 | VIPR1 |
| 2 | NM_002133 | HMOX1 |
| 3 | NM_007061 | HSPA8 |
| 4 | NM_031993 | IRAK1 |
| 5 | NM_000234 | LIG1 |
| 6 | NM_001375 | MAD2L1 |
| 7 | XM_005002 | PCNA |
| 8 | NM_002128 | PLAB |
| 9 | NM_016218 | PRC1 |
| 10 | NM_005410 | SEPP1 |
| 11 | NM_006865 | TNFAIP3 |
| 12 | NM_001071 | TYMS |
| 13 | NM_014501 | UBE2S |
| 14 | NM_022036 | GPRC5C |
| 15 | XM_052673 | MAOA |
| 16 | XM_011126 | STK6 |
| 17 | XM_006181 | HIST1H3J |
| 18 | NM_005573 | LMNB1 |
| 19 | NM_153604 | PRO2000 |
| 20 | NM_005502 | ABCA1 |
| 21 | NM_001706 | BCL6 |
| 22 | NM_020386 | AKR1B10 |
| 23 | NM_021967 | BCL2L1 |
| 24 | NM_007338 | BIRC5 |
| 25 | XM_010017 | CACNG4 |
| 26 | NM_005194 | CCNA2 |
| 27 | NM_003883 | CCNB1 |
| 28 | NM_032969 | CDC20 |
| 29 | NM_005345 | CST3 |
| 30 | NM_147780 | CTSB |
| 31 | NM_000104 | CYP1B1 |
| 32 | NM_001955 | EDN1 |
| 33 | NM_006829 | FANCG |
| 34 | NM_002483 | GGH |
| 35 | NM_002084 | GPX3 |
| 36 | NM_001960 | HMGB1 |
| 37 | NM_002129 | HMGB2 |

TABLE 7A

| Gene | Gene | |
|---|---|---|
| No. | Identifier | Gene Name |
| 1 | NM_022036 | GPRC5C |
| 2 | XM_052673 | MAOA |
| 3 | XM_011126 | STK6 |
| 4 | XM_006181 | HIST1H3J |
| 5 | NM_005573 | LMNB1 |
| 6 | NM_153604 | PRO2000 |
| 7 | NM_001706 | BCL6 |

TABLE 7B

| | Gene | |
|---|---|---|
| Gene No. | Identifier | Gene Name |
| 1 | NM_004354 | CCNG2 |
| 2 | NM_005518 | HMGCS2 |
| 3 | NM_000029 | AGT |
| 4 | NM_198252 | GSN |
| 5 | NM_006341 | MAD2L2 |
| 6 | NM_014397 | NEK6 |
| 7 | NM_004176 | SREBF1 |
| 8 | NM_203401 | STMN1 |
| 9 | NM_006732 | FOSB |
| 10 | NM_032637 | SKP2 |

1. A compound having the structure of Formula I 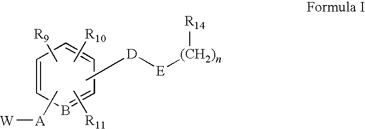
wherein W is one of 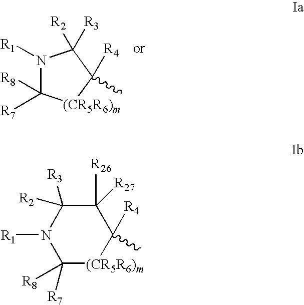
wherein when W is structure Ia, m=0, 1, 2, or 3 and n=0, 1, 2, 3, 4, or 5;
and when W is structure Ib, m=1 or 2 and n=0, 1, 2, 3, 4, or 5;
and wherein A is selected from O and —CR₂₀R₂₁
B is selected from N and —CR₁₂;
D is selected from C═O and —CR₂₂R₂₃,
E is selected from NR₁₃ and —CR₂₄R₂₅,
such that when A is O, if D is C═O then E is —CR₂₄R₂₅ and if E is NR₁₃ then D is —CR₂₂R₂₃,
R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxy, polyaromatic, and 5-7 ring atom heteroaryl with heteroatom N or O,
and wherein R₁ is further selected from the structure —XY,
wherein X═(CR₃₀R₃₁)ₖ (wherein k=0, 1, 2 or 3), SO₂, C═O, NR₃₀R₃₁ or —C(═O)NR₃₀, wherein R₃₀ and R₃₁ are each independently H, CH₃, or C₂ to C₅ alkyl,
and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ alkenyl or C₂-C₄ alkynyl chains that may themselves be substituted or unsubstituted;
and wherein R₁₃ and R₁₄ are each further selected independently from —CHO, OR₁₅, SR₁₅, or NR₁₅R₁₆, C₁-C₄-alkyl-aryl and aryl-C₁-C₄-alkyl;
R₂, R₃, R₄, R₅, R₆, R₇, R₈, R₉, R₁₀, R₁₁, R₁₂, R₁₄, R₂₀, R₂₁ R₂₂, R₂₃, R₂₄, R₂₅, R₂₆, and R₂₇ are each independently selected from H, F, Cl, Br, I, OH, CF₃, CH₃, C₂ to C₅ alkyl, C₂ to C₅ alkenyl, C₁ to C₅ alkoxy, C₁ to C₅ hydroxyalkyl, NR₁₅R₁₆,
and wherein R₁₅ and R₁₆ are each independently selected from H, CH₃, C₂ to C₅ alkyl,
and wherein NR₁₃(CH₂)ₙR₁₄ or a portion thereof may combine to form a substituted or unsubstituted ring selected from piperidine, pyrrolidine, and tetrahydroisoquinoline,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, SO₂NR₁₈R₁₉, CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

2-3. (canceled)

4. The compound of claim 1, wherein R₉ is H, Cl or OMe.

5. The compound of claim 1, wherein R₁ is 4-phenylphenyl, carbazole, dibenzothiophene, dibenzofuran, fluorene, phenyl or naphthyl.

6-17. (canceled)

18. A compound having the structure of Formula II 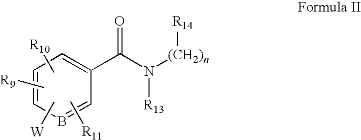
wherein W is one of 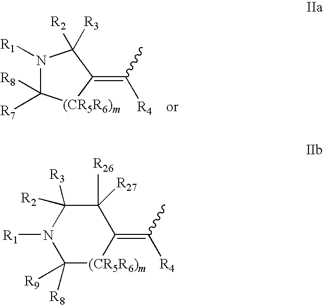
wherein when W is structure IIa, m=0, 1, 2, or 3 and n=0, 1, 2, 3, 4, or 5;
and when W is structure IIb, m=1 or 2 and n=0, 1, 2, 3, 4, or 5;
B is selected from N and —CR₁₂;
R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxy, polyaromatic, and 5-7 ring atom heteroaryl with heteroatom N or O,
and wherein R₁ is further selected from the structure —XY,
wherein X═(CR₃₀R₃₁)ₖ (wherein k=0, 1, 2 or 3), SO₂, C═O, NR₃₀R₃₁ or —C(═O)NR₃₀, wherein R₃₀ and R₃₁ are each H, CH₃, or C₂ to C₅ alkyl, and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ alkenyl or C₂-C₄ alkynyl chains that may themselves be substituted or unsubstituted;
and wherein R₁₃ and R₁₄ are each further selected independently from —CHO, OR₁₅, SR₁₅, or NR₁₆R₁₆, C₁-C₄-alkylaryl and aryl-C₁-C₄-alkyl;
R₂, R₃, R₄, R₅, R₆, R₇, R₈, R₉, R₁₀, R₁₁, R₁₂, R₁₄, R₂₆, and R₂₇ are each independently selected from H, F, Cl, Br, I, OH, CF₃, C₁ to C₅ alkyl, C₁ to C₅ alkenyl, C₁ to C₅ alkoxy, C₁ to C₅ hydroxyalkyl, NR₁₅R₁₆ (wherein R₁₅ and R₁₆ are each independently selected from H and C₁ to C₅ alkyl);
and wherein R₁₅ and R₁₆ are each independently selected from H, CH₃, and C₂ to C₅ alkyl,
and wherein NR₁₃(CH₂)ₙR₁₄ or a portion thereof may combine to form a substituted or unsubstituted ring selected from piperidine, pyrrolidine, and tetrahydroisoquinoline,
and wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, SO₂NR₁₈R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

19-20. (canceled)

21. The compound of claim 18, wherein R₉ is H, Cl or OMe.

22. The compound of claim 18, wherein R₁ is 4-phenylphenyl, carbazole, dibenzothiophene, dibenzofuran, fluorene, phenyl or naphthyl.

23-24. (canceled)

25. A compound having the structure of Formula III 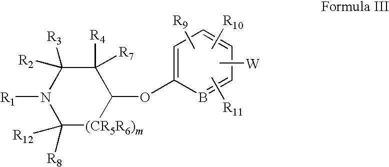
wherein W has the structure 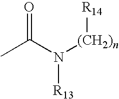
wherein m=0, 1, 2 or 3 and n=0, 1, 2, 3, 4 or 5,
and B is selected from N and —CR₁₂;
and wherein R₁ is further selected from the structure —XY,
wherein X═(CR₃₀R₃₁)ₖ (wherein k=0, 1, 2 or 3), SO₂, C═O, NR₃₀R₃₁ or —C(═O)NR₃₀, wherein R₃₀ and R₃₁ are each H, CH₃, or C₂ to C₅ alkyl, and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ alkenyl or C₂-C₄ alkynyl chains that may themselves be substituted or unsubstituted;
R₂, R₃, R₄, R₅, R₆, R₇, R₈, R₉, R₁₀, R₁₁, R₁₂, and R₁₃ are each independently selected from H, F, Cl, Br, I, OH, CF₃, CH₃, C₂ to C₅ alkyl, C₂ to C₅ alkenyl, C₁ to C₅ alkoxy, C₂-C₄ hydroxyalkyl, NR₁₅R₁₆,
and wherein R₁₃ is further selected from Cᵢ to C₅ hydroxyalkyl and —CHO,
and wherein R₁₄ is selected from H, CH₃, C₂ to C₈ alkyl, branched and unbranched C₂ to C₅ alkenyl, branched and unbranched C₂ to C₅ alkynyl, C₅ to C₇-cycloalkyl, OR₁₅, SR₁₅, —C(═O)R₁₅, —C(═O)OR₁₅, branched and unbranched (C₁ to C₅ alkyl)-NR₁₅R₁₆, NR₁₅R₁₆, branched and unbranched (C₁ to C₅ alkyl)-⁺NR₁₅R₁₆R₁₇, ⁺NR₁₅R₁₆R₁₇,
C(═O)NR₁₅R₁₆, C(═O)ONR₁₅R₁₆, 5 to 7 membered heterocycloalkyl having up to 3 heteroatoms selected from N or O;
aryl, heteroaryl with heteroatom N or O, aralkyl, and alkylaryl,
and wherein each of said cycloalkyl, aryl, heteroaryl and heterocycloalkyl may be further substituted with groups each independently selected from H, F, Cl, Br, I, CF₃, branched and unbranched C₁ to C₅ alkyl, branched and unbranched C₁ to C₅ alkenyl, branched and unbranched C₁ to C₅ alkynyl, branched and unbranched C₁ to C₅ alkoxy, branched and unbranched —C₁ to C₅ alkylamino, branched and unbranched —C₁ to C₅ aminoalkyl, —C(═O)R₁₅, —C(═O)R₂₁, C(═O)OR₁₅, C(═O)OR₂₁, C₅ to C₇-cycloalkyl, —OR₁₅, —SR₁₅, —NR₁₅R₁₆, wherein each of said alkyl, alkenyl, alkynyl, alkoxyl, alkylamino and amino alkyl groups may be further substituted with one or more of methyl, ethyl, n-propyl, isopropyl, n-butyl, sec-butyl, isobutyl or tert-butyl;
and wherein R₁₅ and R₁₆ are each independently selected from H, C₁ to C₅ alkyl, C₁ to C₅ alkyl-R₂₁, C₂ to C₅ alkenyl, substituted or unsubstituted phenyl, —C(═O)R₁₉, —C(═O)OR₁₉, (C₁ to C₅ alkyl)-OH, (C₁ to C₅ alkyl)-NR₁₉R₂₀, —NR₁₉R₂₀,
C(═O)—NR₁₉R₂₀ (wherein each of said R₁₉ and R₂₀ is independently H, methyl, ethyl, n-propyl, isopropyl, n-butyl, sec-butyl, isobutyl or tert-butyl;),
and wherein R₂₁ is selected from 5 to 7 membered cycloalkyl, 5 to 7 membered aryl, 5 to 7 membered heteroaryl, and 5 to 7 membered heterocycloalkyl, wherein said heteroatom is N or O, each of which may be substituted with groups selected from R₁₅,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

26-27. (canceled)

28. The compound of claim 25, wherein R₉ is H, Cl or OMe.

29. The compound of claim 25, wherein R₁ is 4-phenylphenyl, carbazole, dibenzothiophene, dibenzofuran, fluorene, phenyl or naphthyl.

30-32. (canceled)

33. A compound having the structure of Formula IV 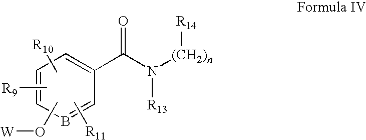
wherein W is one of 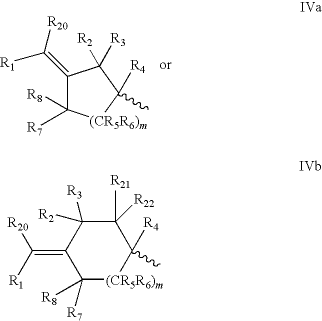
wherein when W is structure IVa, m=0, 1, 2, or 3 and n=0, 1, 2, 3, 4, or 5;
and when W is structure IVb, m=1 or 2 and n=0, 1, 2, 3, 4, or 5;
B is selected from N and —CR₁₂;
R₁₃ and R₁₄ are each independently selected from
H, CH₃, C₂ to C₅ alkyl, C₂ to C₅ alkenyl, C₁ to C₅ alkoxy, 5-9 ring atom cycloalkyl,
OR₁₅, SR₁₅, or NR₁₅R₁₆;
5-9 ring atom heterocycloalkyl having up to 3 heteroatoms selected from N or O and wherein when said heteroatom is N, it may be further substituted as may any carbon in said ring;
5-7 ring atom aryl, aryloxyl, polyaromatic, 5-7 ring atom heteroaryl with heteroatom selected from N and O,
aralkyl and alkylaryl;
R₁, R₂, R₃, R₄, R₅, R₆, R₇, R₉, R₉, R₁₀, R₁₁, R₁₂, R₁₄, R₂₀, R₂₁ and R₂₂ are each independently selected from H, F, Cl, Br, I, OH, CF₃, C₁ to C₅ alkyl, C₁ to C₅ alkenyl, C₁ to C₅ alkoxy, NR₁₅R₁₆;
and wherein R₁₅ and R₁₆ are each independently selected from H, CH₃ and C₂ to C₅ alkyl
and wherein NR₁₃(CH₂)ₙR₁₄ or a portion thereof may combine to form a substituted or unsubstituted ring selected from piperidine, pyrrolidine, and tetrahydroisoquinoline,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

34-35. (canceled)

36. The compound of claim 33, wherein R₉ is H, Cl or OMe.

37. The compound of claim 33, wherein R₁ is 4-phenylphenyl, carbazole, dibenzothiophene, dibenzofuran, fluorene, phenyl or naphthyl.

38-40. (canceled)

41. A compound having the structure of Formula V 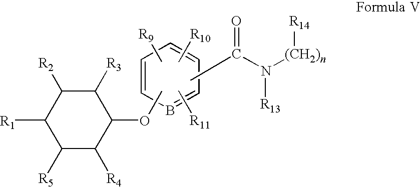
wherein n=0, 1, 2, 3, 4 or 5,
wherein B is selected from N and —CR₁₂;
R₁₃ and R₁₄ are each independently selected from
H, C₁ to C₅ alkyl, C₁ to C₅ alkenyl, C₁ to C₅ alkoxy, 5-9 ring atom cycloalkyl,
OR₁₅, SR₁₅, or NR₁₅R₁₆;
5-9 ring atom heterocycloalkyl having up to 3 heteroatoms selected from N or O and wherein when said heteroatom is N, it may be further substituted as may any carbon in said ring;
5-7 ring atom aryl, aryloxyl, polyaromatic, 5-7 ring atom heteroaryl with heteroatom N or O,
aralkyl and alkylaryl;
R₁, R₂, R₃, R₄, R₅, R₉, R₁₀, R₁₁, R₁₂, R₂₀, R₂₁ R₂₂, R₂₃, R₂₄, R₂₅, R₂₆, and R₂₇ are each independently selected from H, F, Cl, Br, I, OH, CF₃, CH₃, C₂ to C₅ alkyl, ═CH—, C₂ to C₅ alkenyl, C₁ to C₅ alkoxy, NR₁₅R₁₆,
and wherein R₁₅ and R₁₆ are each independently selected from H, CH₃ and C2 to C₅ alkyl
and wherein NR₁₃(CH₂)ₙR₁₄ or a portion thereof may combine to form a substituted or unsubstituted ring selected from piperidine, pyrrolidine, tetrahydroisoquinoline, and piperazine,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-7 ring atom cycloalkyl, 5-7 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

42. The compound of claim 41, wherein each of R₂, R₃, R₄ and R₅ is hydrogen.

43. (canceled)

44. The compound of claim 41, wherein R₉ is H, Cl or OMe.

45-50. (canceled)

51. A composition comprising a therapeutically effective amount of a compound of Formula I, II, III, IV, V or VI in a pharmaceutically acceptable carrier, with substituents as defined herein.

52-56. (canceled)

57. A method of preventing, treating or ameliorating cancer or tumor metastasis in a mammal comprising administering to said mammal an effective amount of a compound of Formula I, II, III, IV, V or VI with substituents as defined herein.

58-71. (canceled)

72. A compound having the structure of Formula VI 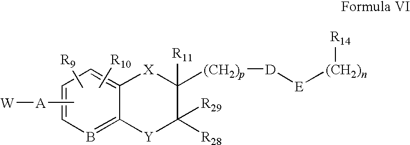
wherein W is one of 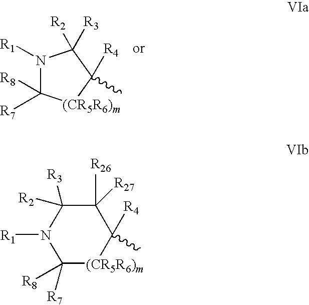
wherein when W is structure VIa, m=0, 1, 2, or 3 and n=0, 1, 2, 3, 4, or 5;
and when W is structure VIb, m=1 or 2 and n=0, 1, 2, 3, 4, or 5;
wherein p=0, 1, 2, or 3;
and wherein A is selected from O and —CR₂₀R₂₁,
B is selected from N and —CR₁₂;
D is selected from C═O and —CR₂₂R₂₃,
E is selected from NR₁₃ and —CR₂₄R₂₅,
X═(CR₃₀R₃₁)_{q}, wherein q=0, 1 or 2,
Y═(CR₃₂R₃₃)ᵣ, wherein r=0, 1 or 2
R₁, R₁₃ and R₁₄ are each selected independently from
H, CH₃, C₂ to C₅ alkyl, C₂ to C₅ alkenyl, C₁ to C₅ alkoxy, 5-9 ring atom cycloalkyl,
5-9 ring atom heterocycloalkyl having up to 3 heteroatoms each independently selected from N or O and wherein when said heteroatom is N, it may be further substituted as may any carbon in said ring;
5-7 ring atom aryl, aryloxy, polyaromatic, and 5-7 ring atom heteroaryl with heteroatom N or O, aralkyl and alkylaryl,
and wherein R₁ is further selected from the structure —XY,
wherein X═(CR₃₀R₃₁)ₖ (wherein k=0, 1, 2 or 3), SO₂, C═O, NR₃₀R₃₁ or —C(═O)NR₃₀, wherein R₃₀ and R₃₁ are each H, CH₃, or C₂ to C₅ alkyl, and wherein Y is selected from a structure containing up to 3 fused or unfused rings of 5 or 6 ring atoms each and each ring is independently selected from aryl, heteroaryl, cycloalkyl and heterocycloalkyl wherein the heteroatom is nitrogen or oxygen, which rings may be substituted or unsubstituted and wherein 2 or all of said rings may be separated by C₁-C₄ alkyl, ═CH—, C₂-C₄ alkenyl or C₂-C₄ alkynyl chains that may themselves be substituted or unsubstituted;
and wherein R₁₃ and R₁₄ are each further selected independently from —CHO, OR₁₅, SR₁₅, or NR₁₅R₁₆
R₂, R₃, R₄, R₅, R₆, R₇, R₈, R₉, R₁₀, R₁₁, R₁₂, R₂₀, R₂₁ R₂₂, R₂₃, R₂₄, R₂₅, R₂₆, R₂₇, R₂₈, R₂₉, R₃₀, R₃₁, R₃₂ and R₃₃ are each independently selected from H, F, Cl, Br, I, OH, CF₃, C₁ to C₅ alkyl, C₁ to C₅ alkenyl, C₁ to C₅ alkoxy, NR₁₅R₁₆
wherein R₁₅ and R₁₆ are each independently selected from H, CH₃ and C₂ to C₅ alkyl;
and wherein NR₁₃(CH₂)ₙR₁₄ or a portion thereof may combine to form a substituted or unsubstituted ring selected from piperidine, pyrrolidine, tetrahydroisoquinoline, and piperazine,
wherein any of said R groups may be substituted or unsubstituted, wherein said substitutions are each independently selected from hydrogen, CH₃, hydroxyl, sulfhydryl, alkoxy, thioalkoxy, alkyl, F, Cl, Br, I, CN, ═O, CF₃, NO₂, 5-9 ring atom cycloalkyl, 5-9 ring atom heterocycloalkyl with 1 or 2 heteroatoms selected from N and O, 5-7 ring atom aryl, 5-7 ring atom heteroaryl with 1 or 2 heteroatoms selected from N and O, alkylaryl, arylalkyl, COOR₁₇, CONR₁₈R₁₉, NR₁₈R₁₉, NR₁₈COR₁₉, NR₁₈SO₂R₁₉, NR₁₇CONR₁₈R₁₉, wherein R₁₇, R₁₈, and R₁₉ are independently as recited for R₂ and wherein each said cycloalkyl, heterocycloalkyl, aryl and heteroaryl may be further substituted with a group selected from R₂;
including all pharmaceutically acceptable salts, derivatives, prodrugs, metabolites, solvates, hydrates, and isomers thereof.

73-77. (canceled)

78. The compound of claim 72, wherein said aryl is phenyl.

79. The compound of claim 72, wherein R₁ is 4-phenylphenyl, carbazole, dibenzothiophene, dibenzofuran, fluorene, phenyl or naphthyl.

80-98. (canceled)
